(19) United States
(12) Patent Application Publication
(10) Pub. No.: US 2020/0060555 A1
(43) Pub. Date: Feb. 27, 2020

(54) MONITORING DEVICES AND METHODS

(71) Applicant: TRUE WEARABLES, INC., Rancho Santa Margarita, CA (US)

(72) Inventors: Marcelo Malini Lamego, Coto de Caza, CA (US); Isadora Buticosky Lamego, Coto de Caza, CA (US); Larissa Buticosky Lamego, Coto de Caza, CA (US)

(21) Appl. No.: 16/551,437

(22) Filed: Aug. 26, 2019

Related U.S. Application Data
(60) Provisional application No. 62/722,676, filed on Aug. 24, 2018.
(60) Provisional application No. 62/723,290, filed on Aug. 27, 2018.

(51) Int. Cl. A61B 5/0205; A61B 5/00; A61B 5/0476; A61B 5/1455

(52) U.S. Cl. CPC A61B 5/0205; A61B 5/742; A61B 5/0476; A61B 5/021; A61B 5/681; A61B 5/746; A61B 5/14551

(57) ABSTRACT

Clinical-grade monitoring technology is disclosed for a number of applications where low-cost, wireless, multi-parameter, single-use and multi-use medical and fitness and/or wellness devices are useful and beneficial. A monitoring device that attaches or is placed on a measurement site includes at least one of an optical sensor, a temperature sensor, or first and second electrical contact sensors. Signals received from the optical sensor, a temperature sensor, and/or first and second electrical contact sensors can be transmitted to a host device. An application program on a host device can process the signals to compute one or more physiological parameters, waveform data, trend data, and/or one or more reports.

### CROSS-REFERENCE TO RELATED APPLICATION(S)

This application claims priority to U.S. Provisional Application No. 62/722,676 entitled “Monitoring Devices and Methods,” filed on Aug. 24, 2018, and to U.S. Provisional Application No. 62/723,290 entitled “Monitoring Devices and Methods,” filed on Aug. 27, 2018, of which each entire disclosure is hereby incorporated by reference in its entirety.

### BACKGROUND

Devices that determine various health parameters are used regularly by consumers and medical personnel. For example, the measurement of blood oxygen saturation (SpO2), pulse rate (PR), and perfusion index (PI) are health parameters consumers and medical personnel monitor to receive feedback on the user's health and/or fitness.

### SUMMARY

Embodiments disclosed herein enable clinical-grade monitoring technology in a number of applications where low-cost, wireless, multi-parameter, single-use and multi-use medical and fitness and/or wellness devices are useful and beneficial. Wireless characteristics enable convenience, comfort, and/or freedom of movement for the user and/or patient. The interplay between single-use and multi-use designs enables use-case flexibility. In medical applications, single-use designs reduce the risks of cross contamination and healthcare associated infections, simplifies workflow, and eliminates failures due to equipment wear and tear. In fitness and wellness applications, multi-use designs enable more affordable solutions for personal use. Embodiments of the monitoring technology can be applied to a number of clinical settings and fitness and wellness applications, including pulse oximetry for COPD, anesthesia, aviation and sports, and oxygen therapy, non-invasive continuous blood glucose monitoring for diabetes disease management, continuous body temperature monitoring, ECG spot-check monitoring, electroencephalogram (EEG) continuous monitoring, non-invasive monitoring of water in blood for body hydration management, non-invasive total hemoglobin monitoring for anemia and/or blood transfusion management, continuous dyshemoglobinemia monitoring, among many others.

In one aspect, a monitoring device includes an optical sensor, a temperature sensor, a first electrical contact sensor, and a second electrical contact sensor within an enclosure of the monitoring device. The optical sensor includes a light source and a photodetector positioned adjacent a first surface of the enclosure. The light source is operable to emit light towards the measurement site and the photodetector is operable to receive light reflected from the measurement site when the first surface is in contact with a measurement site of a first body part of a user. The temperature sensor is positioned adjacent the first surface of the enclosure and is operable to measure a temperature at the measurement site when the first surface is in contact with the measurement site. The first electrical contact sensor is positioned adjacent the first surface of the enclosure to be in contact with the measurement site when the first surface is in contact with the measurement site. The second electrical contact sensor is positioned adjacent a second surface of the enclosure. The first and the second electrical contact sensors detect heart signals when a different second body part of the user contacts the second electrical contact sensor. The monitoring device also includes a wireless communication device that is operable to transmit signals received from the photodetector, temperature measurements, and the heart signals to an application program on a host device.

In another aspect, a system includes a monitoring device and an application program on a host device. The monitoring device includes an optical sensor, a temperature sensor, a first electrical contact sensor, and a second electrical contact sensor within an enclosure of the monitoring device. The optical sensor includes a light source and a photodetector positioned adjacent a first surface of the enclosure. The light source is operable to emit light towards the measurement site and the photodetector is operable to receive light reflected from the measurement site when the first surface is in contact with a measurement site of a first body part of a user. The temperature sensor is positioned adjacent the first surface of the enclosure and is operable to measure a temperature at the measurement site when the first surface is in contact with the measurement site. The first electrical contact sensor is positioned adjacent the first surface of the enclosure to be in contact with the measurement site when the first surface is in contact with the measurement site. The second electrical contact sensor is positioned adjacent a second surface of the enclosure. The first and the second electrical contact sensors detect heart signals when a different second body part of the user contacts the second electrical contact sensor. The monitoring device also includes a wireless communication device that is operable to transmit signals received from the photodetector, temperature measurements, and the heart signals. The application program on the host device is operable to process the signals transmitted by the monitoring device to compute a physiological parameter, waveform data associated with the physiological parameter, and trend data associated with the physiological parameter, and cause the physiological parameter and at least one of a waveform associated with the physiological parameter or a trend associated with the physiological parameter to be displayed.

In one embodiment, the monitoring device is a discrete device that is attached to a measurement site on a user. In another embodiment, the monitoring device is incorporated into a hat, such as a baseball hat, that is worn by the user. The hat can include built-in electroencephalogram (EEG) electrodes. Alternatively, the monitoring device is incorporated into a patch that attaches to the measurement site. The hat and the patch can include circuitry that process signals, transmit the signals wirelessly to a host device, and perform operations such as power management and energy harvesting.

The monitoring device can obtain measurements for various physiological parameters continuously or at select times and transmit the measurements to a host device. An application program on the host device can display a measurement gauge for one or more physiological parameters, a body temperature gauge, one or more waveforms, and/or trend waveforms or charts in a user interface or screen of the application program. The application program can generate an alarm when a physiological parameter exceeds an upper limit and/or is below a lower limit. The upper and lower limits can be set in a settings user interface of the application program.

In some aspects, the battery life of the battery in the monitoring device can be estimated by the application program on the host device, by the monitoring device, or in a distributed process using both the host device and the monitoring device. The computations in are performed in both a closed loop and an open loop via different functions. In the closed loop, the battery voltage and/or other available parameters of interest (e.g., ambient temperature, circuitry loads, etc.) is used to estimate the battery charge directly. In the open loop, the battery charge is estimated indirectly using one or more counters and/or other available parameters of interest (e.g., ambient temperature, circuitry loads, etc.).

### BRIEF DESCRIPTION OF THE DRAWINGS

Non-limiting, and non-exhaustive examples are described with reference to the following Figures. The elements of the drawings are not necessarily to scale relative to each other. Identical reference numerals have been used, where possible, to designate identical features that are common to the figures.

FIG. 1 is a block diagram illustrating an example of a monitoring device connected to a computing device and network;

FIGS. 2A-2F depict example measurement sites on a patient's body, where a monitoring device can be applied;

FIGS. 2G-2L illustrate example adhesive tape layouts suitable for use with a monitoring device;

FIGS. 2M-2O depict example measurement sites on a patient's body, where a monitoring device can be applied'

FIG. 3 depicts an example of a modulation scheme that is suitable for use with a monitoring device;

FIGS. 4A-4C illustrates an example of another modulation scheme that is suitable for use with a monitoring device;

FIG. 5 is a cross-section view of an example of a monitoring device;

FIGS. 6A-6C are schematic diagrams depicting an example of a monitoring device;

FIG. 7 is a flow chart illustrating an example method of processing measurement data received from a monitoring device;

FIG. 8A depicts an example embodiments of a monitoring device with an optical sensor;

FIG. 8B illustrates an example embodiment of a monitoring device with a temperature sensor and an optical sensor;

FIG. 8C depicts an example embodiment of a monitoring device with an optical sensor and electrical contact sensors;

FIG. 8D illustrates an example embodiment of a monitoring device with an optical sensor, electrical contact sensors, and a temperature sensor;

FIG. 8E depicts an example embodiment of processing circuitry configured to process signals received from the temperature sensor and/or the electrical contacts shown in FIGS. 8A-8D;

FIG. 9A depicts the application program that works with the monitoring device installed in a host device;

FIGS. 9B-9C illustrate the monitoring device and accessories;

FIG. 9D details the steps to activate the monitoring device;

FIGS. 9E-91 depict a workflow showing several exemplary embodiments for attaching the monitoring device to various measurement sites;

FIGS. 9J-9Q illustrate the steps to start the application program in a host device, connect the monitoring device to the host device, and begin continuous data exchange between monitoring device and the host to produce measurements and waveforms;

FIGS. 9R-9T depict exemplary embodiments for a wearable fitness/medical hat;

FIG. 9U depicts exemplary embodiments for a wearable fitness/medical patch;

FIGS. 10A-10B depict a workflow to share data collected, processed and analyzed by a host device that works in combination with the medical, and fitness and wellness device technology;

FIGS. 10C-10L show exemplary charts with analyses from data collected from a user wearing the medical, and fitness and wellness device technology;

FIGS. 10M-10N show exemplary file formats for storing trends and encoded data format waveforms;

FIGS. 100-10P detail the steps to compute the log-volatility and volatility of measurement data;

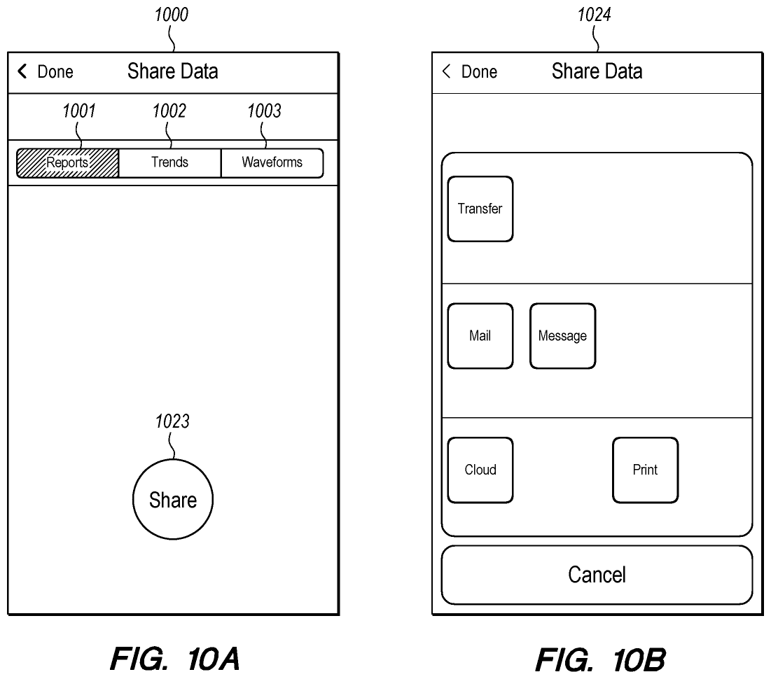

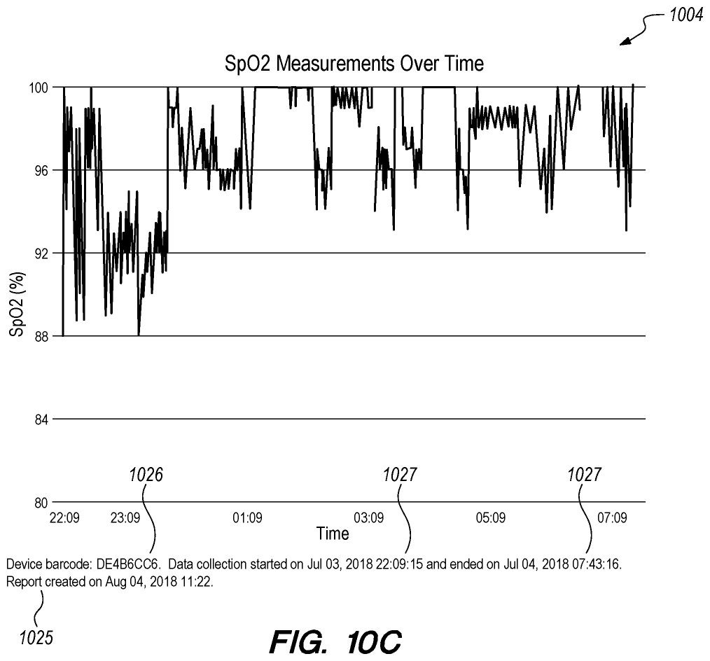

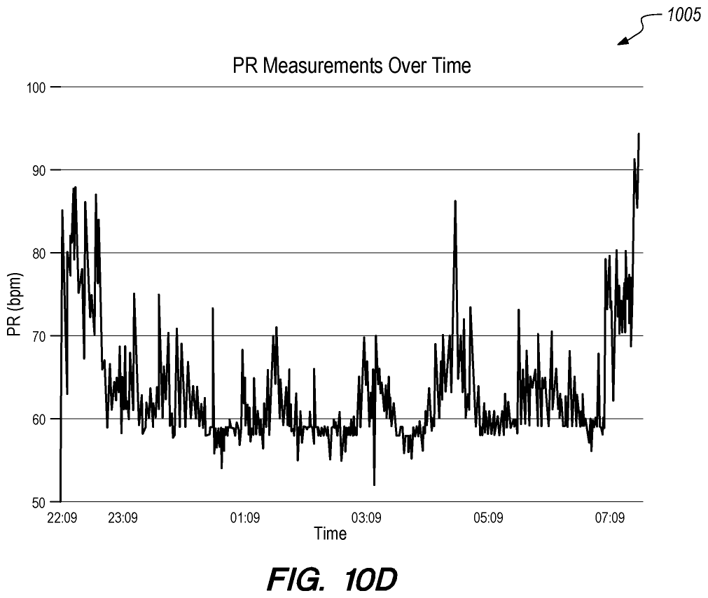

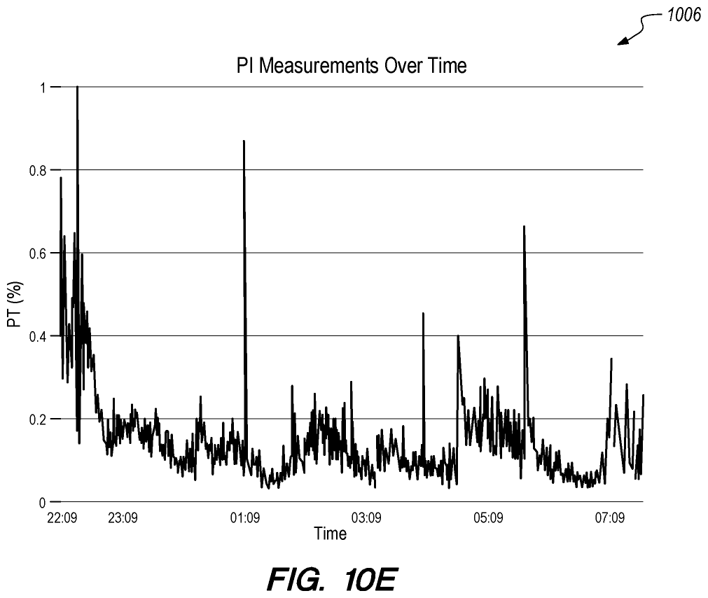

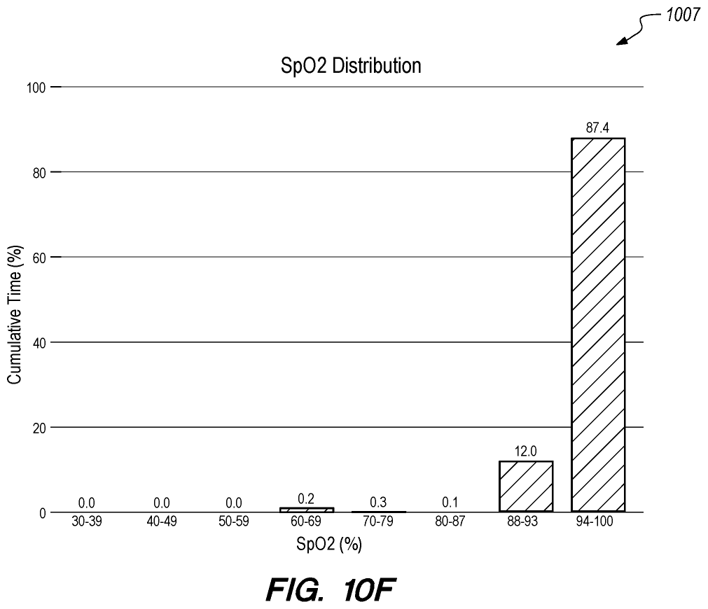

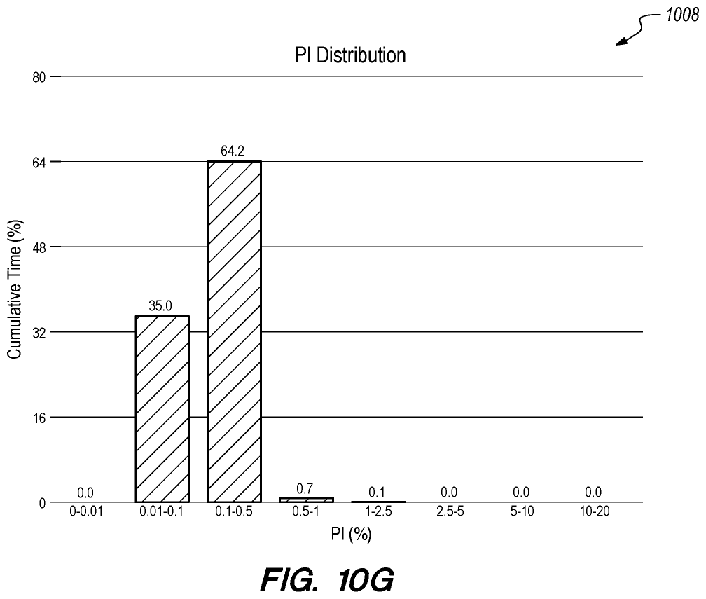

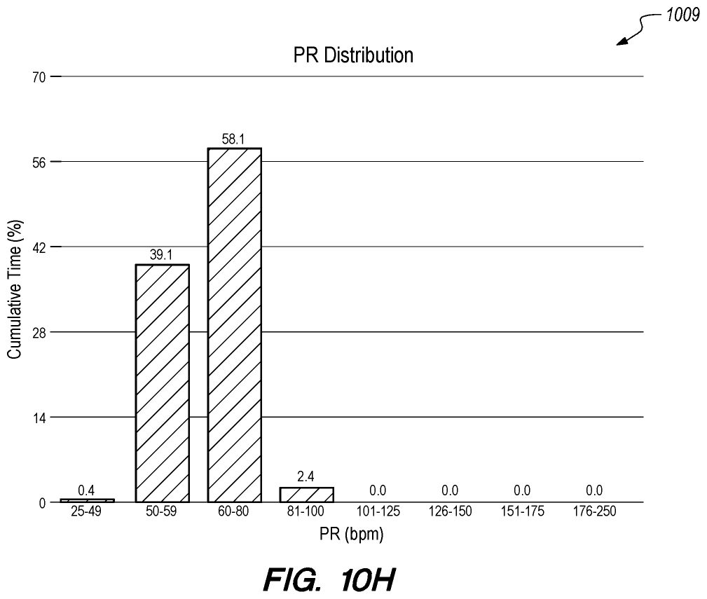

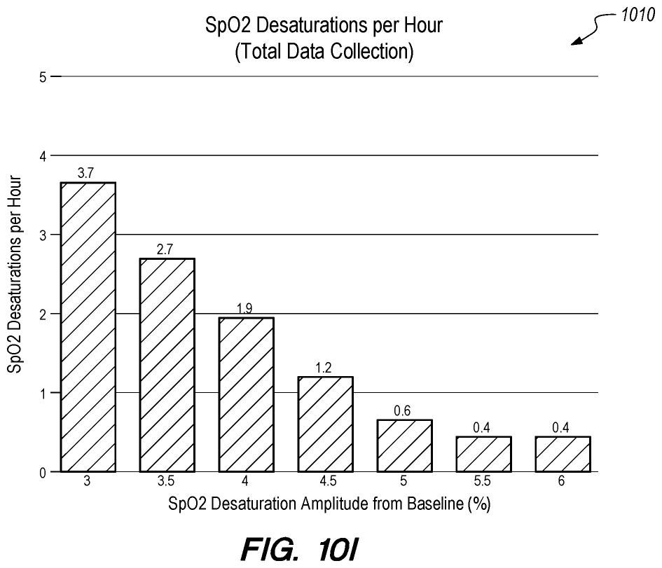

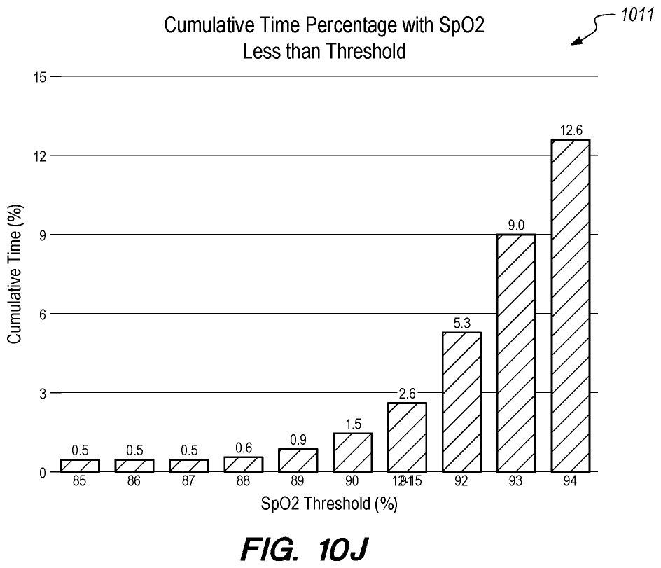

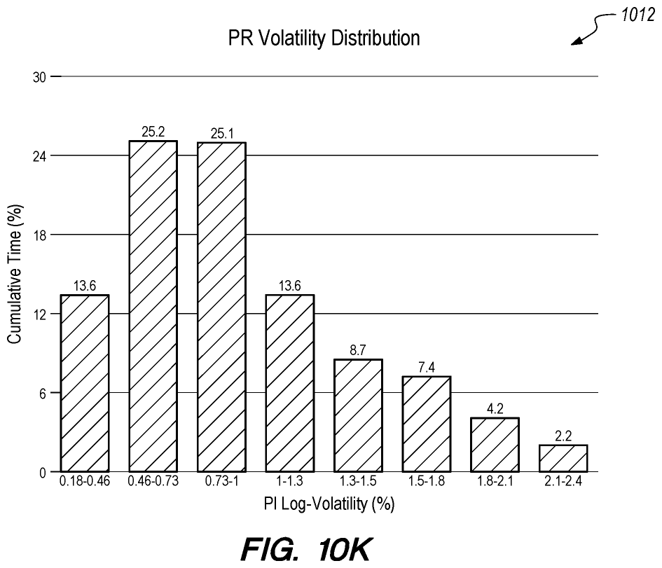

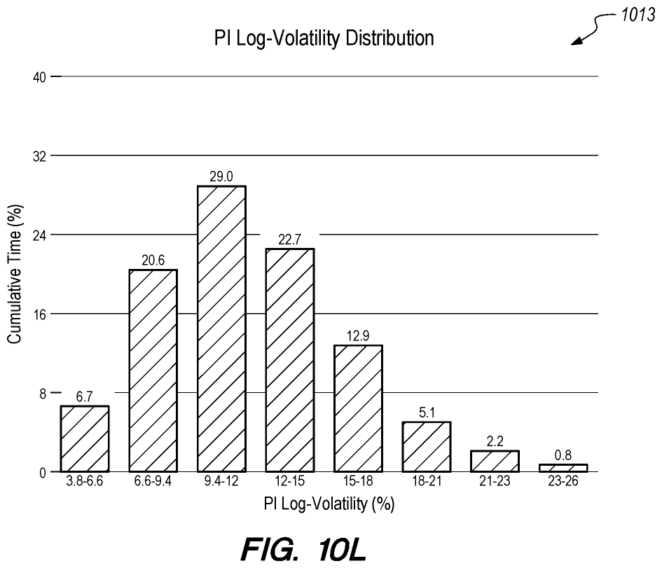

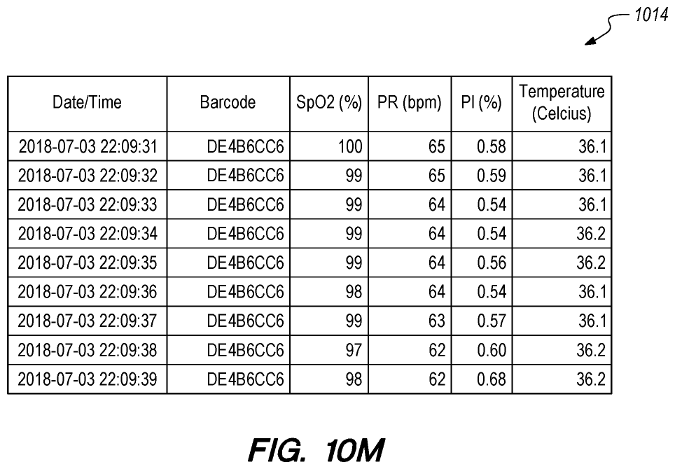

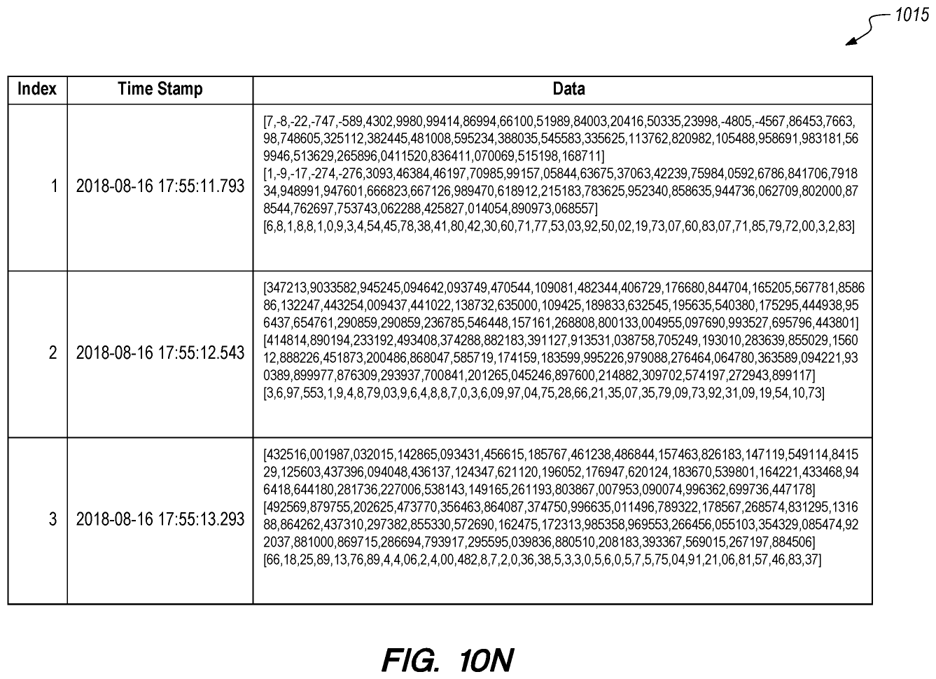

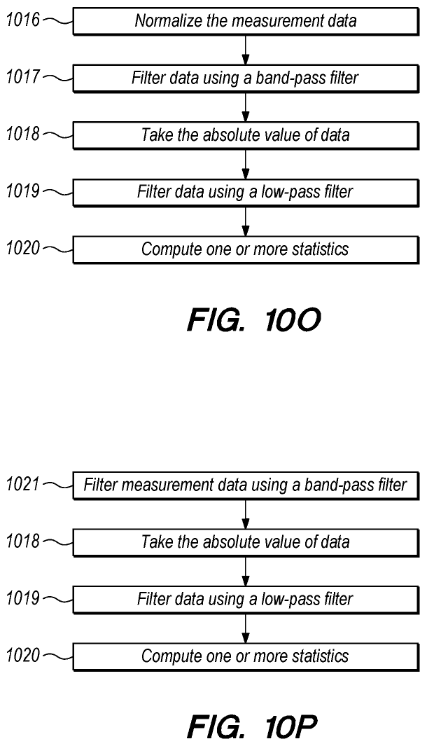

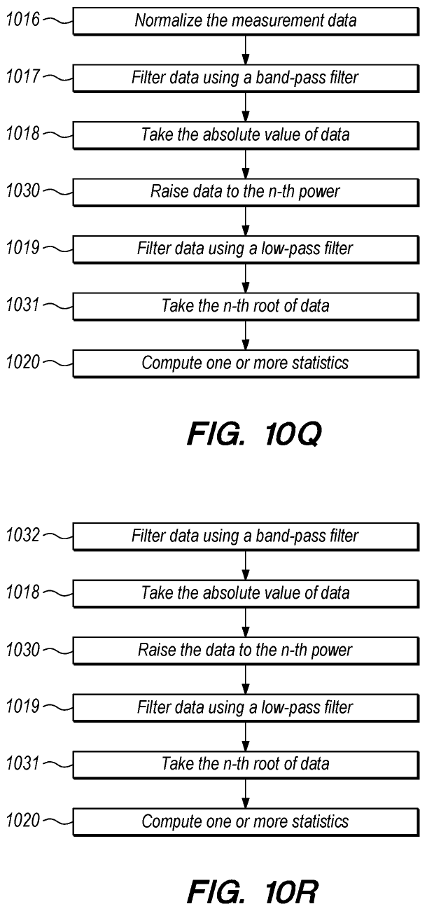

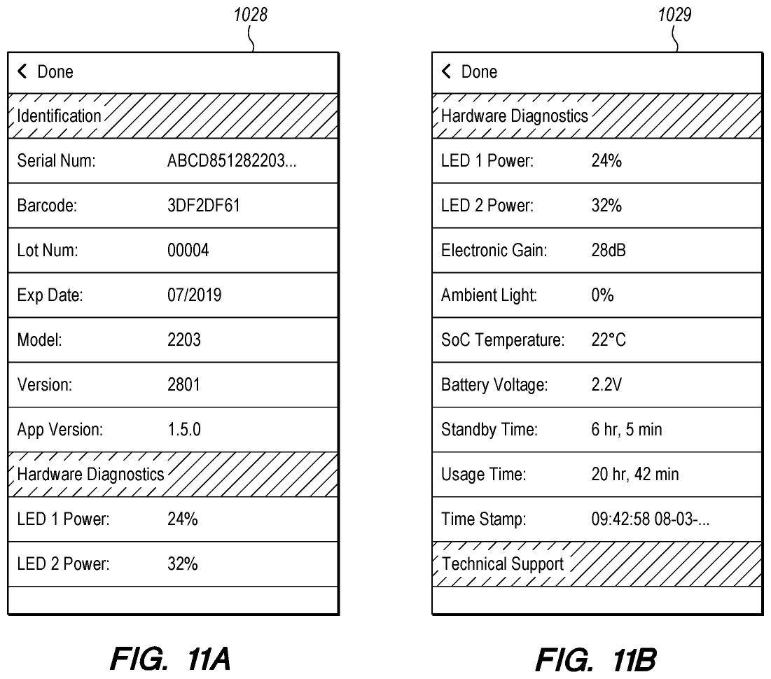

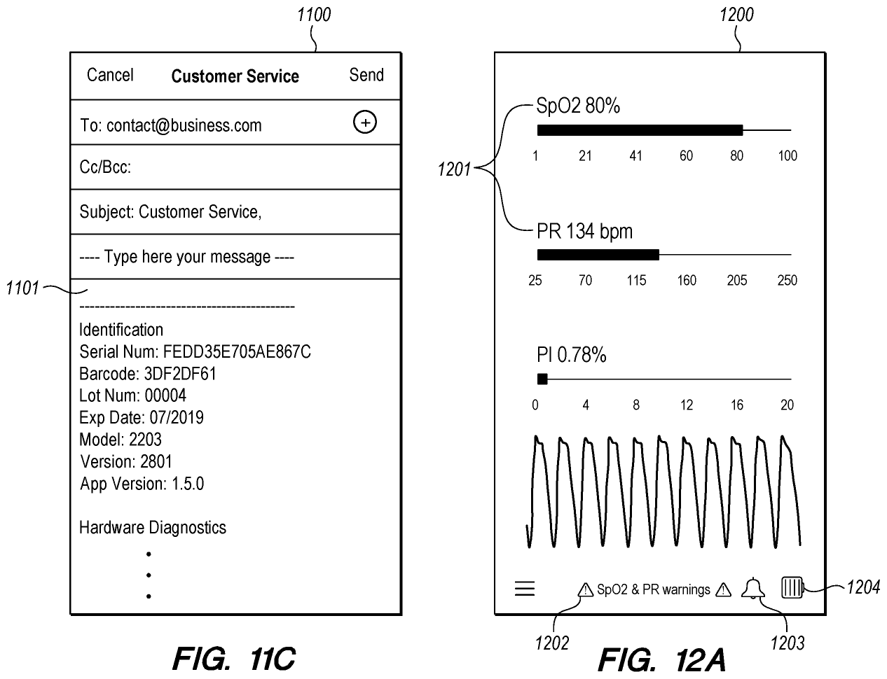

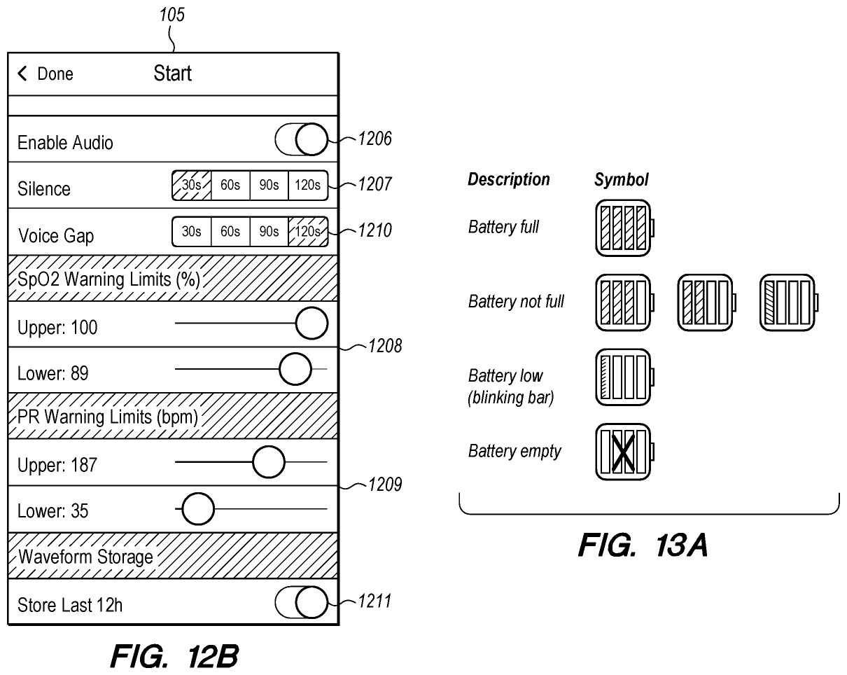

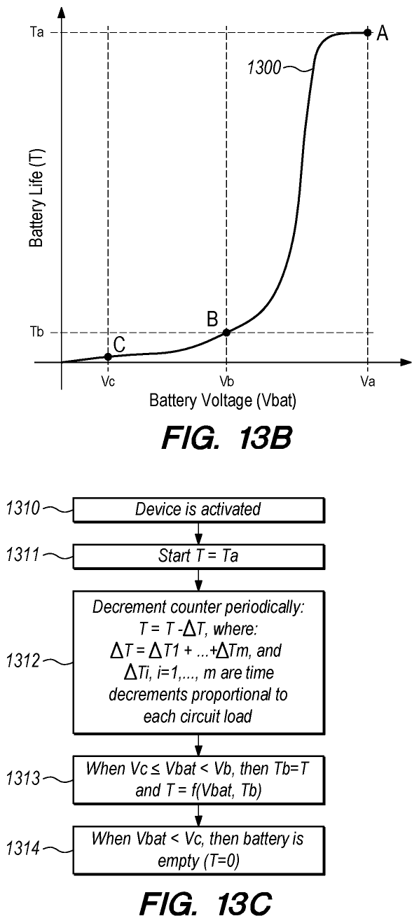

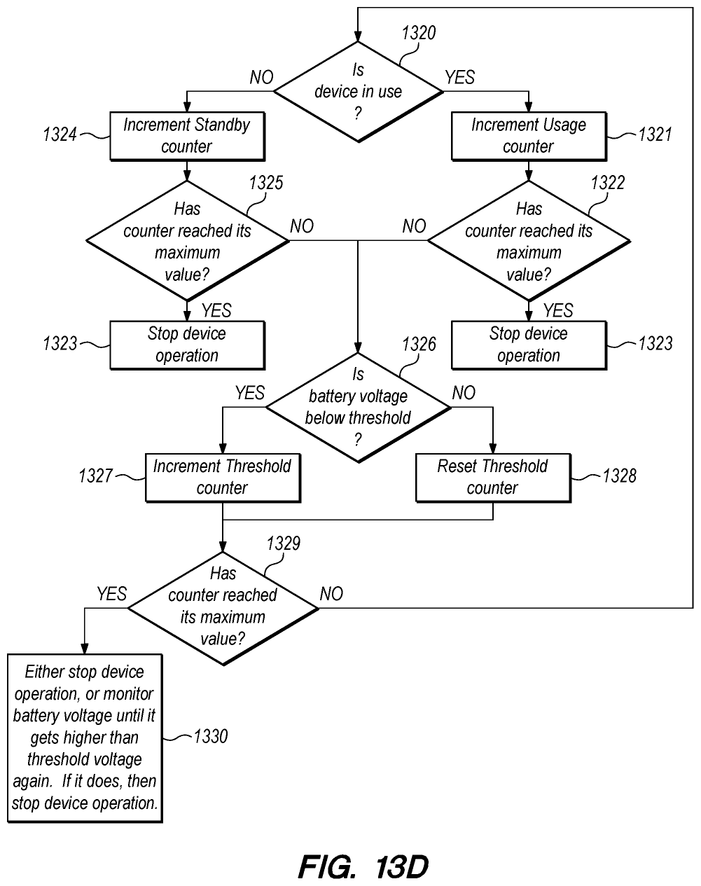

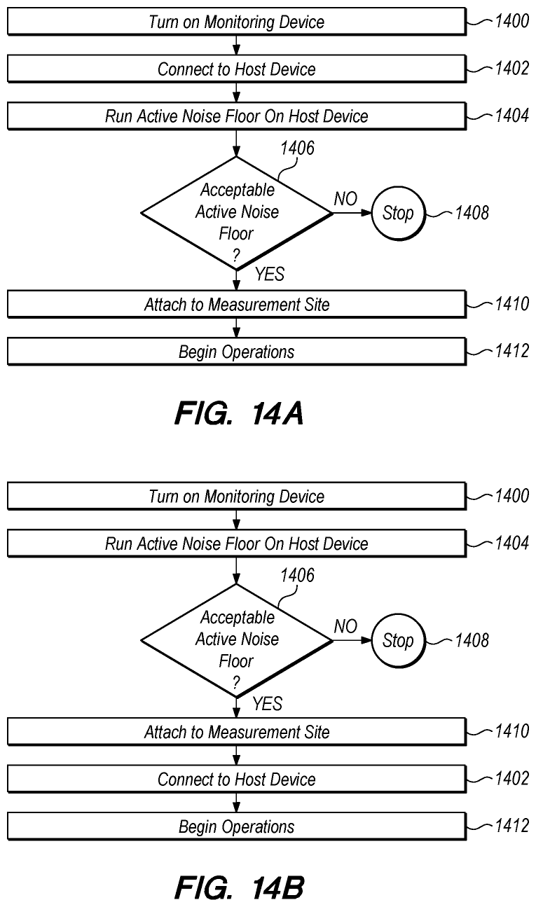

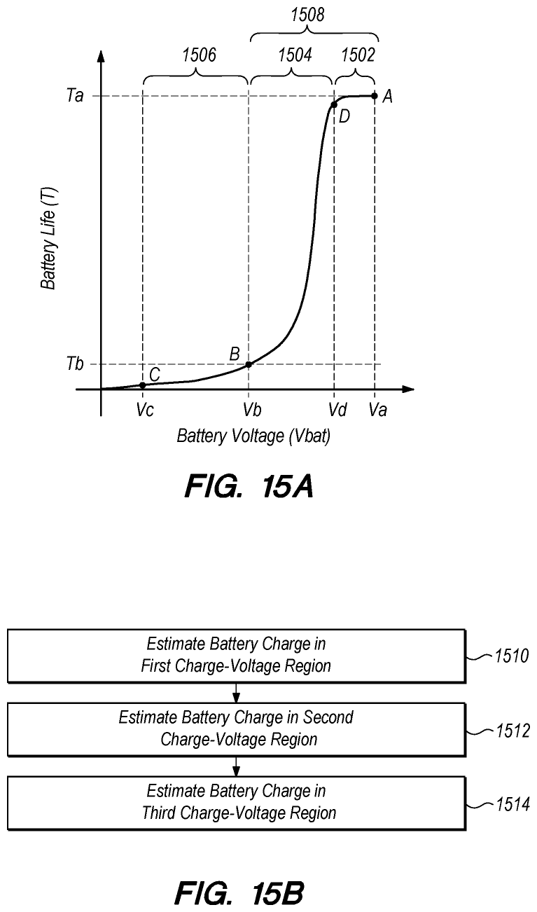

FIGS. 10Q-10R detail the steps to compute the Ln log-volatility and Ln volatility of measurement data;

FIGS. 11A-11B show an embodiment where identification and hardware diagnostics parameters are produced by the application program on the host device;

FIG. 11C depicts an example embodiment on sharing the measurement data with a technical support team of the monitoring device;

FIG. 12A depicts an example embodiment of an alarm/warning system;

FIG. 12B illustrates an example settings screen of the application program;

FIG. 13A depicts a battery icon and example battery states;

FIG. 13B illustrates an example battery discharge curve;

FIG.13C depicts an example method of estimating the battery life of the battery in the monitoring device;

FIG. 13D depicts a flowchart of an anti-tampering method for the monitoring device;

FIG. 14A depicts a first example method of determining an active noise floor of a monitoring device;

FIG. 14B illustrates a second example method of determining an active noise floor of a monitoring device;

FIG. 15A depicts an example battery discharge curve;

FIGS. 15B-15C illustrate example methods of estimating the battery life of the battery in the monitoring device; and

FIG. 16 illustrates a flowchart of a method of operating a monitoring device.

### DETAILED DESCRIPTION

As used herein, the term “optimal” is intended to be construed broadly and is intended to cover values and models that provide best, substantially best, and acceptable values and models. As used herein, the term “data stream” refers to data sequentially indexed by an exogenous quantity (e.g., time, space, etc.). For instance, data streams that are function of time, are assumed to be indexed by time such as in a discrete-time system. Data streams that are function of space, are assumed to be indexed by space. Depending on the application, data streams can be indexed by quantities that have physical meaning or that are abstract in nature. The embodiments disclosed herein can be applied to any data stream regardless of its indexing or sampling method.

Reference will now be made in detail to representative embodiments illustrated in the accompanying drawings. It should be understood that the following descriptions are not intended to limit the embodiments to the described example embodiments. As used herein, a monitoring device is an electronic fitness or monitoring device that measures, tracks, and/or reports data regarding measurements of one or more physiological parameters, including, but not limited to, heart rate, blood perfusion, oxygen saturation, body temperature, and the like. The measurements or data relating to the measurements are sent to a computing device for further processing. For example, oxygen saturation (SpO2), pulse rate (PR), and perfusion index (PI) can be estimated on a computing device.

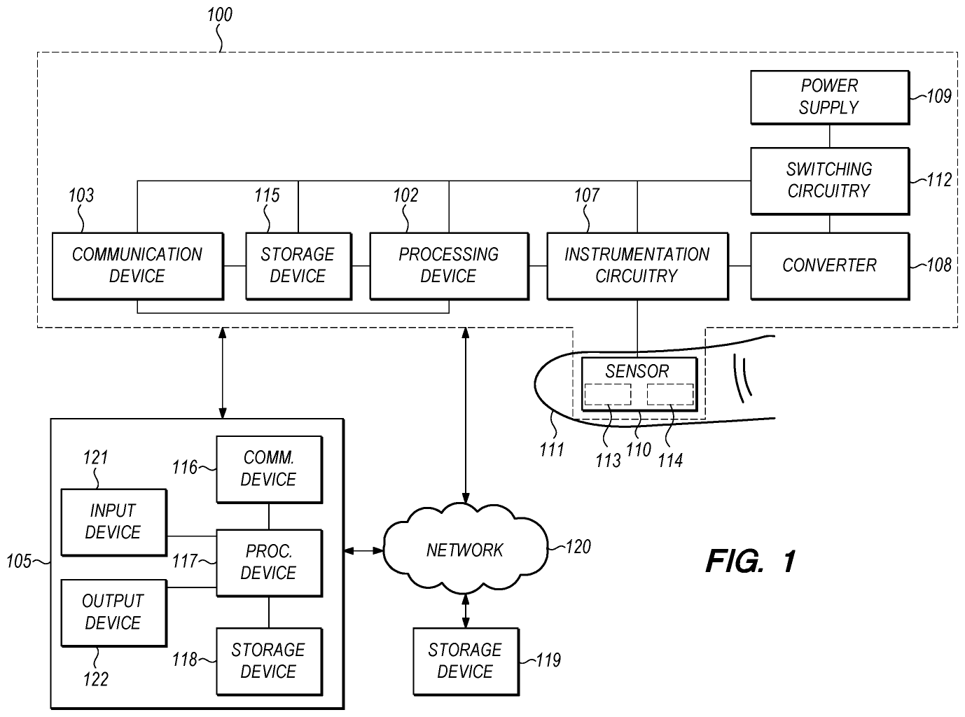

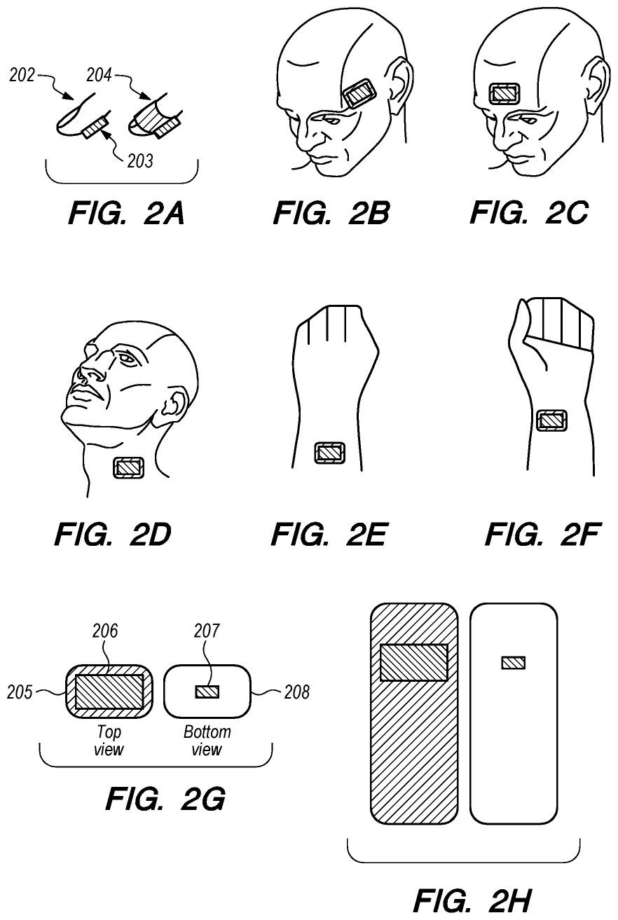

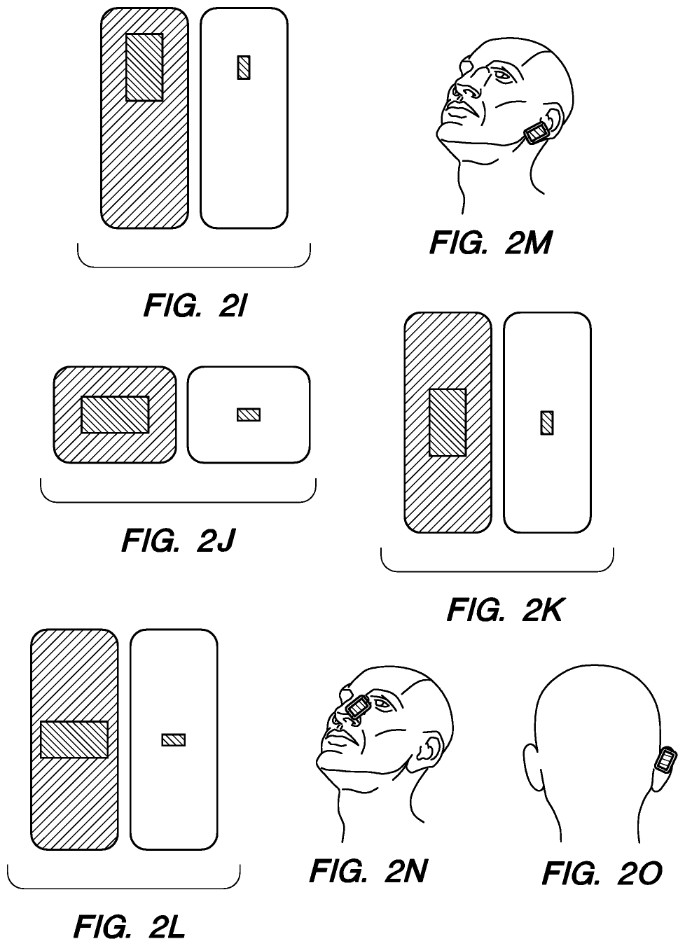

FIG. 1 is a block diagram illustrating an example of a monitoring device connected to a computing device and network. A monitoring device 100 is attached to one or more measurement sites from which the sensor can readily access blood perfusion information. In the illustrated embodiment, the measurement site 111 is a finger or digit (see also FIG. 2A). Other example measurement sites include, but are not limited to, a patient's temple (FIG. 2B), forehead (FIG. 2C), neck (FIG. 2D), arm (FIGS. 2E and 2F), ear or ear lobe (FIG. 2M), nose (FIG. 2N), and/or the posterior auricle of an ear (FIG. 2O).

In one embodiment, the monitoring device 100 includes a processing device 102, instrumentation circuitry 107, a communication device 103, and a storage device 115. The instrumentation circuitry 107, the communication device 103, and the storage device 115 are connected to the processing device(s) 102. A converter 108 is connected to the instrumentation circuitry 107 and switching circuitry 112. The communication device 103, the storage device 115, the processing device 102, and the instrumentation circuitry 107 along with a power supply 109 are connected to the switching circuitry 112. The monitoring device 100 may also include adhesive tape to attach the monitoring device 100 to a measurement site.

The monitoring device 100 may be turned on using the switching circuitry 112. In one instance, the switching circuitry 112 is a single-use, conductive tape-switch. The instrumentation circuitry 107 may include one or more light sources, such as a light-emitting diode (LED), control circuitry and logic, and one or more photodetectors, such as a photodiode. The communication device 103 can be any suitable type of communication device, including, but not limited to, a wireless low energy radio (examples of which include, but are not limited to, BLE, ANT, Zigbee, etc.). Wireless connection and authentication (when required) between communication devices 116 and 103 can be accomplished through standard pairing methods (i.e., Just Works, etc.) and out-of-band methods, such as Near-Field Communication (NFC), barcode/image scanning, or via an optical link between the optical sensor 110 and camera (or optical sensor) housed in the computing device 105. Depending on the configuration of the monitoring device 100, the storage device 115 may comprise, but is not limited to, volatile storage (e.g., random access memory), non-volatile storage (e.g., read-only memory), flash memory, or any combination of such memories.

The monitoring device 100 can be communicatively coupled to a computing device 105, such as a smart phone, a tablet computing device, a desktop or laptop computer, a wireless computing and/or data aggregator appliance device, a bedside monitor, or a similar computing device through a wired or wireless connection. The computing device 105 can include a communication device 116 and a storage device 118 connected to a processing device 117. The monitoring device 100 transmits, via the communication device 103, measurement data to the computing device 105 (via the communication device 116) to be processed, displayed and/or stored. The measurement data can be used for alarms, for electronic medical record data transfer, for data sharing, and/or for other uses of the data.

The computing device 105 may further include one or more input devices (represented by input device 121) and/or one or more output devices (represented by output device 122). The input and output devices 121, 122 are connected to the processing device 117. The input device 121 can be implemented as any suitable input device, such as a keyboard (physical or virtual), a mouse, a trackball, a microphone (for voice recognition), an image capture device, and/or a touchscreen or touch display, or any other computer-generated perceptual input information. The output device 122 may be implemented as any suitable output device, such as a display, one or more speakers, and/or a printer, or any other computer-generated perceptual output information. In some embodiments, the measurement data or data representative of the measurement data can be provided to the output device 122. For example, the measurement data or data representative of the measurement data may be displayed on a display.

In some embodiments, the computing device 105 and/or the monitoring device 100 can access an external storage device 119 through one or more networks (represented by network 120) to store and/or retrieve measurement data. In one or more embodiments, the network 120 is illustrative of any suitable type of network, for example, an intranet, and/or a distributed computing network (e.g., the Internet) over which the computing device and/or the monitoring device 100 may communicate with other computing devices.

As will be described in more detail later, the measurement data produced by the monitoring device 100 can be processed to determine or estimate one or more physiological parameters (e.g., pulse rate, blood oxygen saturation). As part of the processing, one or more signals are processed with a numerical solver device. The numerical solver device can be implemented with one or more circuits (circuitry), a software algorithm or program executed by one or more processing devices (e.g., processing device 102 and/or processing device 117), or a combination of circuitry and a software algorithm.

For example, in one embodiment, the storage device 115 in the monitoring device 100 can include a number of software programs or algorithms and data files, including a numerical solver device. While executing on the processing device 102, the numerical solver device may perform and/or cause to be performed processes including, but not limited to, the aspects as described herein. In another embodiment, the storage device 118 in the computing device 105 can include a number of software programs or algorithms and data files, including a numerical solver device. While executing on the processing device 117, the numerical solver device may perform and/or cause to be performed processes including, but not limited to, the aspects as described herein. In yet other embodiments, the operations of the numerical solver device are distributed such that some of the operations are performed by the processing device 102 and some of the operations are performed by the processing device 117.

FIGS. 2G-2L illustrate example adhesive tape layouts suitable for use with a monitoring device. In the figures, each monitoring device uses a different adhesive tape layout, such as those depicted in FIGS. 2G and 2H, respectively. Specifically, in the illustrated embodiment, a monitoring device 203 is attached to a patient's fingertip 202 using a flat adhesive bandage 205 encapsulated in a polytetraftuoroethylene (PTFE) pocket or in an origami made out of biocompatible tape 206, as shown in FIG. 2G. An optical sensor 207 on the underside 208 of the monitoring device 203 may contact the patient's skin when the underside 208 is adhered to the patient's skin. In some of the many alternative embodiments, such as those shown in FIGS. 2H through 2L, an adhesive bandage or tape 204 may be used to attach the monitoring device 203 to a measurement site.

Small Footprint

Embodiments of the monitoring device can provide a relatively small footprint (size). Among other aspects, a smaller size can require less material in manufacturing, improved ease of use, less room required for storage, less costs for transport, and a less intrusive device and instrument for patients' increased comfort and mobility when using the monitoring device. In one embodiment, the monitoring device 100 may include a printed circuit board (PCB) comprising the processing device 102 with an integrated communication device 103, a compact integrated circuit that includes the instrumentation circuitry 107 for signal conditioning and LED current driving, the power supply 109, and the converter 108. The power supply 109 combined with the converter 108 provide the required higher voltage to drive the light sources 113 and/or a photodetector 114 of the optical sensor 110. In one embodiment, the converter 108 is a single DC-DC switched converter, the power supply 109 is a disposable battery, the light sources 113 are LEDs, and the photodetector 114 is a silicon photodiode.

The processing device 102 and the instrumentation circuitry 107 can be powered directly by the power supply 109. The optical sensor 110 may be encapsulated with the PCB by any one of a wide variety of suitable apparatus and methods, including (by way of example) by attaching flexible adhesive tapes of various types (optionally combined with PTFE) to the PCB. Persons of ordinary skill in the art will understand that the PCB may be rigid or flexible, or be in the form of a substrate where some or all the components are die attached and wire bonded to the substrate, and encapsulated for protection using epoxy or some other encapsulation material. Further, the optical sensor 110 may be attached to a measurement site 111 using any of a wide variety of suitable apparatus and methods, including (by way of example) by using the adhesive tapes that are part of the optical sensor 110 encapsulation structure (as described herein).

Low Power Consumption

In some aspects, the processing device 102 is a low-power ARM processor with dual functionality for controlling a wireless low energy radio (communication device 103) and instrumentation circuitry 107. The optical sensor 110 can include high efficiency LEDs and at least one silicon photodiode that are arranged in a reflective configuration such that the LEDs and the at least one silicon photodiode are physically separated from each other to minimize the required LED currents and frontend gains in the instrumentation electronics. The instrumentation circuitry 107 may have very low bias currents and operate at low voltages. In one embodiment, ambient light interferences may be avoided or at least reduced by modulating and time-multiplexing the LEDs' currents at a higher frequency to shift the spectral content of the generated and detected optical signals to a range in the spectrum where ambient light interferences are less likely to occur.

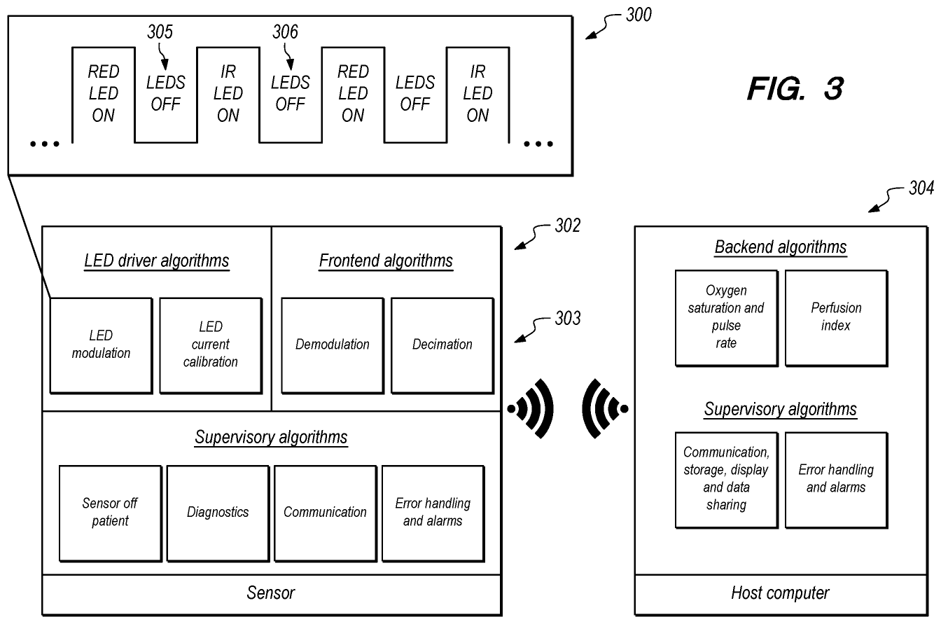

FIG. 3 depicts a distributed system suitable for processing signals produced by a monitoring device and an example modulation scheme that is suitable for use with a monitoring device. The modulation scheme 300 may reduce the complexity of the demodulation, decimation, LED current calibration, sensor off patient, error handling and alarms, diagnostics, and/or communication algorithms shown in the algorithm block diagram 302. Some or all of the blocks in the algorithm block diagram 302 are included in a monitoring device 303. The LED driver algorithms, the frontend algorithms, and the supervisory algorithms can each be software programs that are stored in the storage device 115.

In the modulation scheme depicted in FIG. 3, each LED (light source 113) is kept turned on for approximately 25% of the modulation time cycle (LED duty cycle). Smaller LED duty cycles can be used to reduce overall power consumption. The LEDs can be kept turned off for approximately 50% of the modulation time cycle. The intervals where the LEDs are turned off can also be increased if the LED duty cycles are to be reduced and if the modulation frequency is kept the same. The two slots 305, 306 in the waveform represent times when the LEDs are turned off. The two slots 305, 306 can be used to probe and cancel the effects of ambient light. Modulation frequencies as low as 1 KHz can be adopted with signal-to-noise ratio figures similar to medical-grade pulse oximeters in embodiments that perform sophisticated filtering and signal processing in the demodulation scheme to recover the optical signals generated by the interplay of the LEDs optical signals and the attenuation caused by the measurement site's blood perfused tissue.

In some embodiments, a distributed computing architecture may be used to calculate one or more physiological parameters such as blood oxygen saturation (SpO2), a pulse rate (PR), and a perfusion index (PI). For example, the SpO2, PR and PI are estimated on a host computing device 304 (e.g., a mobile phone or laptop) to increase the battery life of the monitoring device. In one embodiment, one or more numerical solver devices can be included in the backend algorithms of the host computing device 304. For example, a numerical solver device can be included in the oxygen saturation and pulse rate algorithm and in the perfusion index algorithm. In another example, one or more numerical solver devices can be a separate algorithm that is called by the oxygen saturation and pulse rate algorithm and by the perfusion index algorithm.

In other embodiments, one or more numerical solver devices can be included in the monitoring device 303. For example, one or more numerical solver devices may be implemented in the frontend algorithms, such as, for example, the demodulation algorithm.

A processing device in the monitoring device 303 (e.g., processing device 102 in FIG. 1) may execute the time critical, high frequency, low latency and low complexity tasks. Data processed by the processing device in the monitoring device 303 may be reduced in bandwidth by decimation algorithms, and sent wirelessly to the host computing device 304 (e.g., to a processing device). In one embodiment, the host computing device 304 may execute more complex, high latency tasks to calculate and continuously display the measurement values for SpO2, PR and PI.

In an example embodiment, the monitoring device frontend from Texas Instruments (AFE4403) can be used as the instrumentation circuitry 107 (FIG. 1). In such embodiments, the monitoring device frontend may be programmed to generate and control directly the required LED modulation scheme without the need for additional resources from the sensor processing device 102.

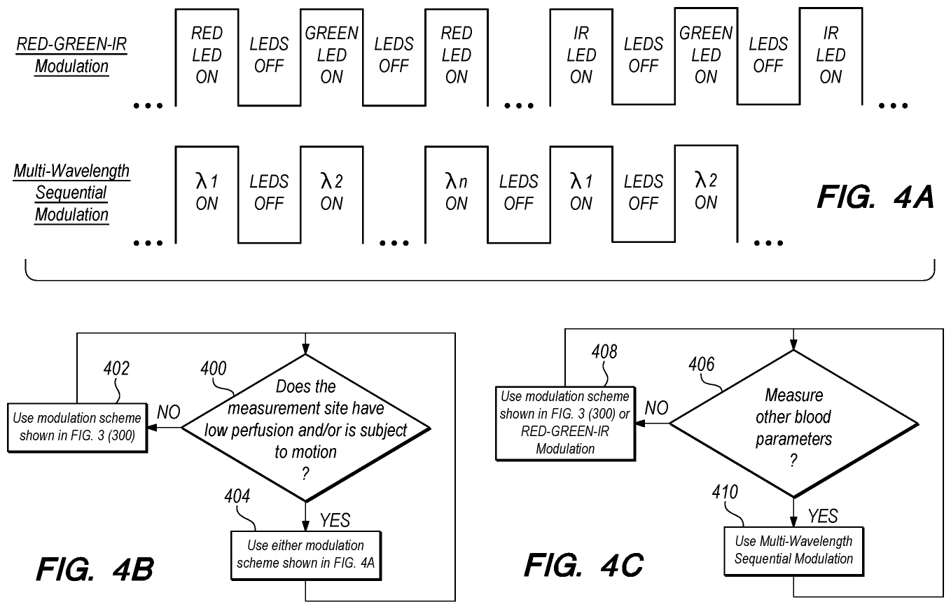

Other example modulation schemes are shown in FIG. 4A. The RED-GREEN-IR Modulation scheme and/or the Multi-Wavelength Sequential Modulation scheme can be used with measurement sites that have low perfusion and/or subject to excessive motion. FIG. 4B and FIG. 4C depict flowcharts of methods of determining which modulation scheme to use. FIGS. 4B-4C show example scenarios where a particular type of modulation can be advantageous. In the method shown in FIG. 4B, the adopted modulation scheme depends on the aforementioned factors (e.g., low perfusion and/or subject to excessive motion). Initially, as shown in block 400, a determination is made as to whether the measurement site has low perfusion and/or is subject to motion. If not, the process passes to block 402 where the modulation scheme shown in FIG. 3 can be used. When the measurement site has low perfusion and/or is subject to motion, the method continues at block 404 where a RED-GREEN-IR Modulation scheme or a Multi-Wavelength Sequential Modulation scheme may be used.

In the method shown in FIG. 4C, a determination is made at block 406 as to whether one or more measurements of other blood parameters are to be obtained or determined. The blood parameters can include, but are not limited to, glucose, water, and hemoglobin. If one or more measurements of other blood parameters is to be determined, the process passes to block 408 where the modulation scheme shown in FIG. 3 or the RED-GREEN-IR Modulation scheme shown in FIG. 4A can be used. If one or more measurements of other blood parameters will not be determined, the method continues at block 410 where the Multi-Wavelength Modulation scheme may be used.

For the RED-GREEN-IR Modulation scheme shown in FIG. 4A, green and red LEDs are activated and modulated for a period of time according to the on-off pattern described, and then, the red LED (RED) is replaced with the near-infrared LED (IR) and also modulated for a period of time. This sequence of events repeats itself while the measurement site is subject to motion and/or low perfusion levels. When light in the wavelength range between violet and yellow (i.e., between 400 to 590 nm approximately) is applied to a blood-perfused measurement site, the higher light scattering and absorption seen in this region, create photoplethysmographs that are much larger in amplitude when compared to the ones in the red and near-infrared wavelength regions. Typically, the green wavelength is used because LEDs in this range offer good efficiency and reliability as well as lower cost when compared to other wavelengths in the violet-yellow range. Also, the optical properties of blood in the green region are desirable in terms of scattering and absorption levels. The photoplethysmograph associated with the green LED can be used to improve detection of the heart rate and/or the detection of the red and near-infrared true photoplethysmograph amplitudes and waveforms, which are required for an accurate measurement of the blood's oxygen saturation under low-perfusion and motion conditions.

The Multi-Wavelength Sequential Modulation scheme shown in FIG. 4A can be used in some embodiments when the parameters of interest require other wavelengths in addition to the red and near-infrared LEDs. Examples include the non-invasive measurement of other blood constituents (parameters), such as glucose, for diabetes disease management, water, for body hydration management, total hemoglobin, for anemia and/or blood transfusion management, etc. As shown in FIG. 4A, a number of light sources of various centroid wavelengths (i.e., λ1, λ2, . . . , λn LEDs) are turned on and off sequentially over time. In the case of the non-invasive measurement of glucose, multiple LEDs in the range of 900 nm to 1700 nm can be adopted. In the case of the non-invasive measurement of total hemoglobin and/or water, wavelengths in the range of 600 nm to 1350 nm should be sufficient. The spectral ranges defined are sufficient because blood and bloodless components at the measurement site have spectral features that are typically quite distinctive depending on the wavelength sub-range under consideration. For instance, water and glucose have higher absorption in the 1550 nm to 1700 nm range than the other components, the hemoglobin species have pronounced features in the 600 nm to 1350 nm, fat has in general pronounced scattering properties throughout the whole range when compared to other blood components, and so on and so forth. The modulation schemes shown in FIG. 3 and FIG. 4A can be switched over time depending on the particular application and/or measurement conditions.

In some embodiments, the method shown in FIG. 4C can be used in a multi-parameter monitoring device that continuously measures SpO2, PR and PI, using the modulation shown in FIG. 3 and/or the RED-GREEN-IR Modulation scheme shown in FIG. 4A. The monitoring device can perform lower-frequency periodic spot-check measurements of other blood parameters, such as the ones previously mentioned (i.e., glucose, water, etc.) using the Multi-Wavelength Modulation scheme. Such a topology is possible because typically water, glucose, hemoglobin, etc. concentrations in blood vary slower when compared to SpO2, PR and PI. Because typical measurement periodicity for the said parameters is in general much longer (i.e., once every 30 minutes, once an hour, etc.), the increase in the monitoring device power consumption is not significant. The additional LEDs and photodetector technologies (i.e., silicon and indium gallium arsenide photodiodes for the 600 nm to 1700 nm wavelength measurement range) required represent small incremental cost and negligible increase in sensor footprint.

The Multi-Wavelength Modulation scheme shown in FIG. 4A can also be used to measure SpO2, PR and PI. In this configuration, the red and near-infrared LEDs are combined with other wavelengths to create “n” photoplethysmographs that can be used to improve SpO2 accuracy or motion performance. Accuracy is improved at least in part because additional LEDs throughout the visible and near-infrared range enable estimation algorithms to counter the optical interference effects of other blood and bloodless components not needed in the measurement of oxygen saturation, pulse rate and/or perfusion. Operation under motion is improved because the effects of motion acceleration on the venous and capillary blood creates optical interferences in the measurement site that have distinct morphological features depending on the wavelength range, and hence are more likely to be eliminated from the photoplethysmographs through advanced signal processing such as the numerical solver device described herein.

Persons of ordinary skill in the art will understand that the wavelengths and other measurements and ranges discussed herein are generally intended to be representative of certain embodiments of the inventions, and not as delimiting as to the many ways in which the inventions can be practiced.

As described earlier, a distributed computing architecture may be used to compute SpO2, PR and PI, where SpO2, PR and PI are estimated on a host computing device (e.g., host computing device 304 in FIG. 3 and computing device 105 in FIG. 1) to increase the monitoring device's battery life. The processing device in the monitoring device (e.g., processing device 102 in FIG. 1) may execute the time critical, high frequency, low latency and low complexity tasks. Data processed by the processing device in the monitoring device may be reduced in bandwidth by decimation algorithms, and sent wirelessly to the host computing device. In one embodiment, one or more processing devices in the host computing device (e.g., processing device 117 in the computing device 105) may execute more complex, high latency tasks to calculate and continuously display the measurement values for SpO2, PR and PI.

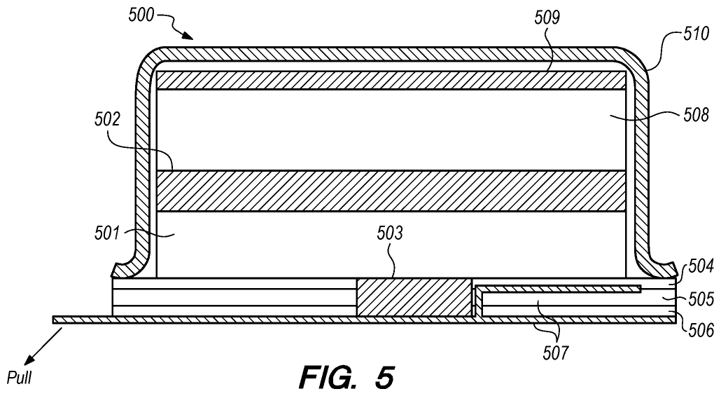

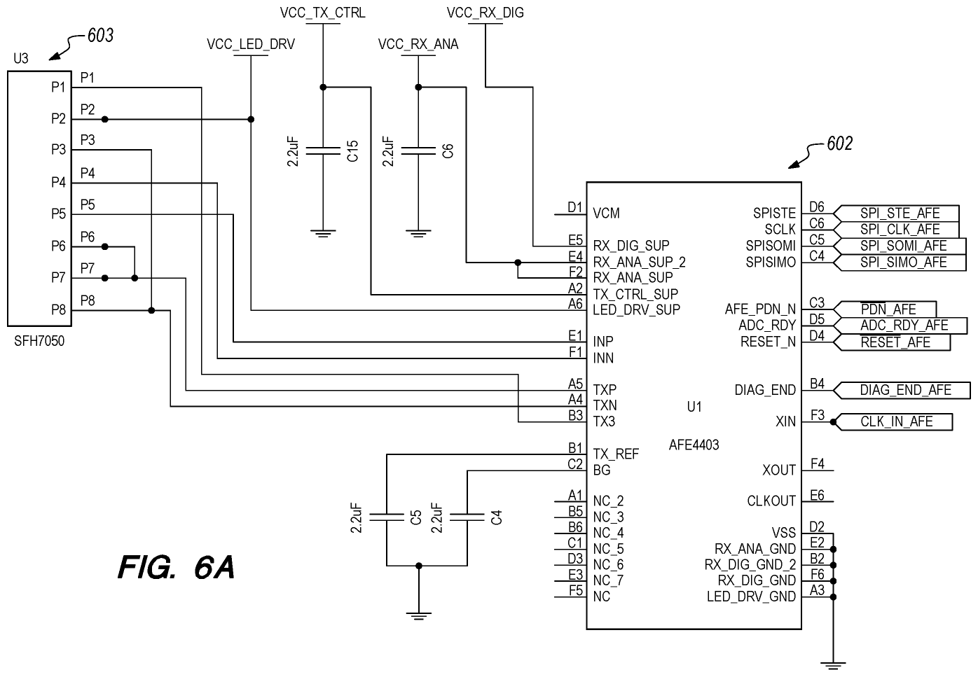

FIG. 5 is a cross-section view of an example monitoring device. FIG. 5 depicts one of the many ways of fabricating a stack-up of the various components of a wireless, disposable, continuous monitoring device 500. A PTFE encapsulation pocket or an origami made out of biocompatible tape 510 may house the components of the monitoring device 500. From top-down, the monitoring device 500 may include: an antenna 509, a battery 508, a printed circuit board (PCB) 501 and PCB circuitry 502, and an optical sensor 503. For attachment to a patient's measurement site such as a fingertip, the monitoring device 500 may include a PCB-to-skin adhesive layer 506. Adhesive layer 505 is made out of electrically conductive tape (such as an isotropically conductive pressure sensitive tape), and adhesive layer 504 contains electrical contacts that (when closed) feed power to PCB 501. A release liner 507 may be disposed between the adhesive layers 504 and 505, and on adhesive layer 506 such that when the release liner is pulled, it exposes the optical sensor 503 and the adhesive layer 506 for attachment to a patient's measurement site, as well as connecting layers 504 and 505, to power on the monitoring device.

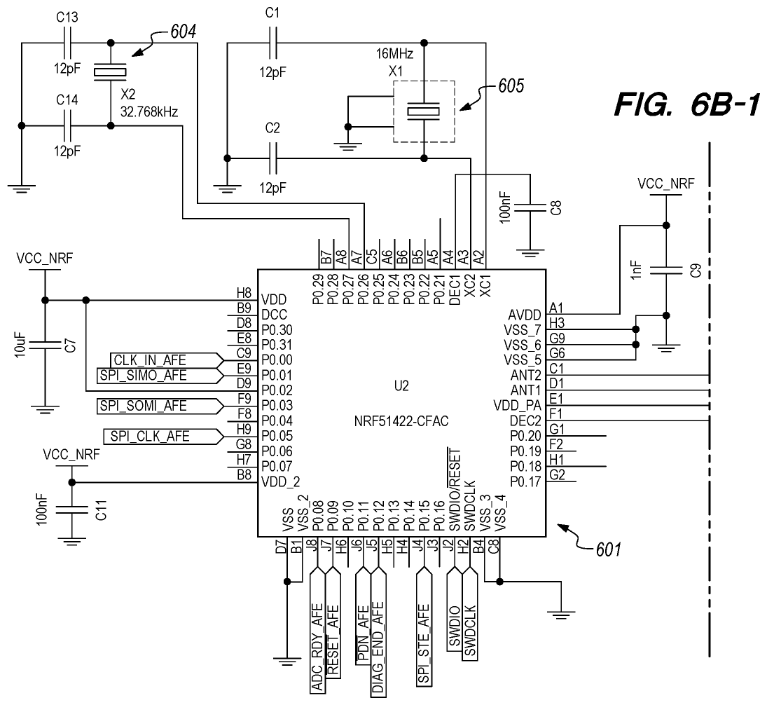

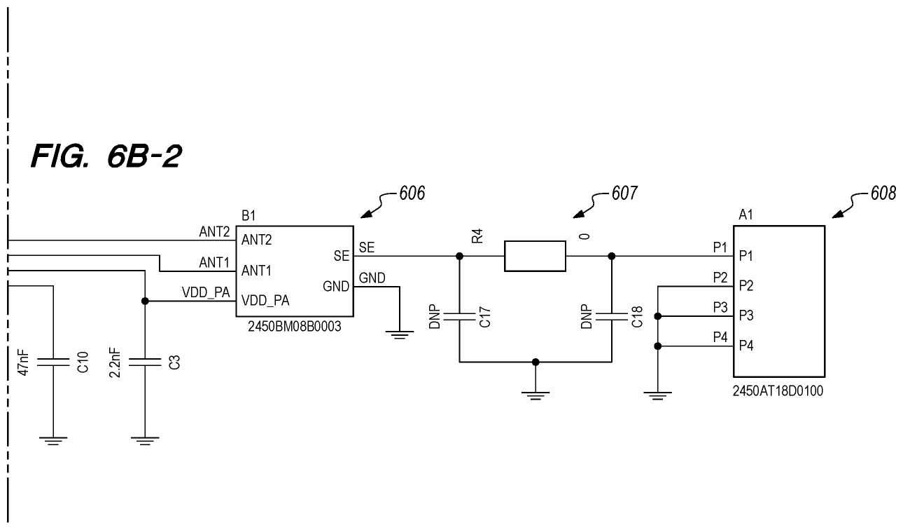

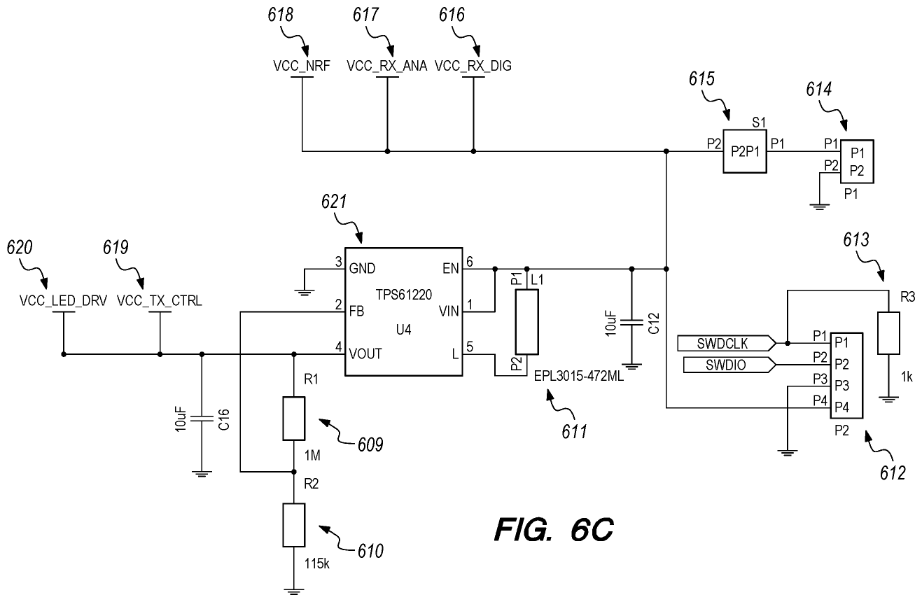

FIGS. 6A-6C are schematic diagrams illustrating an example monitoring device. The monitoring device may include an integrated circuit 602 (FIG. 6A) such as the AFE4403 or the AFE4490 circuits by Texas Instruments, including a photodiode frontend, LED drivers, and control logic. An optical sensor 603 (FIG. 6A), such as the SFH7050 sensor by OSRAM, may include green, red, and near-infrared LEDs and a silicon photodiode. The monitoring device may include a main processing device 601 (FIG. 6B), such as the ARM Cortex MO processor available from Nordic Semiconductors. Further, the monitoring device may include a 16 MHz crystal oscillator 605, a 32.768 kHz crystal oscillator 604 (when ANT low-energy radio is used), a 2.45 GHz impedance balloon filter (single to differential) 606, a matching impedance circuit 607, and an antenna 608 (FIG. 6B). A power management circuit of the monitoring device shown in FIG. 6C may include: a boost converter 621 such as TPS61220 from Texas Instruments, a ferrite inductor 611, boost converter voltage setting resistors 609, 610, debug pads 612 for the main processing device 601, noise rejection pull down resistor 613, battery voltage terminals 614, ON switch pads 615, and voltages 616, 617, 618, 619, 620 for the main processing device 601(FIG. 6B) and integrated circuits. In one embodiment, the ON switch pads 615 are single use pads.

As should be appreciated, the components depicted in FIGS. 6A-6C, and the corresponding descriptions of FIGS. 6A-6C, are for purposes of illustration only and are not intended to limit embodiments to a particular sequence of steps or a particular combination of hardware or software components.

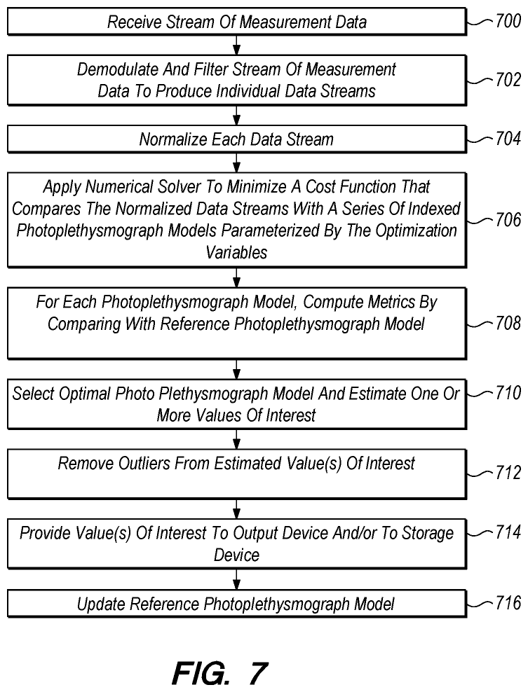

FIG. 7 is a flowchart illustrating an example method of processing measurement data received from a monitoring device. The illustrated method fits measurement data to a model and based on the model, determines one or more physiological parameters (e.g., PR, SpO2, PI). Depending on the application, the method of FIG. 7 is performed once, or the method repeats for a given number of times. For example, with a monitoring device, the method shown in FIG. 7 can repeat as long as a stream of measurement data is received. In a non-limited example of a monitoring device, the method of FIG. 7 repeats substantially every 0.75 seconds.

Initially, as shown in block 700, a stream of measurement data is received. In one embodiment, the stream of measurement data is a time-multiplexed and modulated digital stream of measurement data. In a monitoring device embodiment, the stream of measurement data represents any suitable number of measurement samples that are captured by the monitoring device at a given sampling frequency (e.g., 4 kHz). In one embodiment, the stream of measurement data is captured continuously by the monitoring device, although other embodiments are not limited to this implementation.

The stream of measurement data is then demodulated and filtered at block 702 to produce individual data streams for each wavelength channel (e.g., red, infrared, etc.). Any suitable demodulation technique can be used. In a non-limiting example embodiment, a demodulation system can include a multi-channel symmetric square wave demodulator device operably connected to a filter device, as disclosed in co-pending U.S. application Ser. No. 16/198,550 filed on Nov. 21, 2018. The filter device can be implemented as a single stage or a multi-stage filter device. In some embodiments, the demodulator device and/or the filter device perform decimation, where the sampling frequency is reduced to a lower value (e.g., from 4 kHz to 1 kHz, from 1 kHz to 50 Hz) to reduce signal processing requirements, wireless bandwidth, and/or power consumption. Additionally or alternatively, the demodulation system and techniques are capable of removing most or substantially all interference signals within a pre-defined continuous frequency range (i.e., 0 Hz to 800 Hz).

In some aspects, each individual data stream is a photoplethysmograph data stream. At block 704, each individual data stream is normalized. In one embodiment, a log of each data stream is taken and bandpass filtered to produce a photoplethysmograph data stream for each wavelength channel.

Next, at block 706, the photoplethysmograph data streams are processed by a numerical solver device to calculate or estimate optimization variables that minimize a cost function to produce one or more photoplethysmograph models. In one embodiment, the photoplethysmograph data streams are processed in data batches of any size that is suitable for a particular application. For example, with a monitoring device, the data batch size can be the equivalent of a few seconds of data (e.g., 250 samples collected over 5 seconds) and updated in real-time every certain time interval (e.g., 0.75 seconds).

In one aspect, the numerical solver can compare the data streams with a series of indexed photoplethysmograph models parameterized by the optimization variables. For example, for each Pulse Rate (PR) value, from 25 to 250 BPM in steps of 1 BPM, the numerical solver device calculates values for the optimization variables that minimize a cost function to produce the best photoplethysmograph model for the given data streams. As disclosed in co-pending U.S. application Ser. No. 16/198,504 filed on Nov. 21, 2018, in one embodiment, the cost function can be defined by the following equation:

J(x, z)=Σᵢ₌₁ⁿ(zᵢAx−bᵢ)^{T}(zᵢAx−bᵢ), Equation 1

where A ∈ R^{k×m}, k≥m, is a constant matrix, bᵢ ∈R^{k}=1,2, . . . n are constant vectors, x ∈ R^{m} and z=[z₁ z₂. . . zₙ]^{T} ∈Rⁿ are the optimization variable vectors, and the T superscript is the transpose operator.

Each photoplethysmograph model produced based on the numerical solver device and its corresponding PR value is considered a data point (pair). As a result, in this example, the photoplethysmograph models are indexed by the PR values. If the cost function is given by Equation 1, then each photoplethysmograph model is represented by the vector Ax and scaling factors zᵢ, and the photoplethysmograph data streams are represented by the vectors bᵢ. The optimization variables are the vector x and scaling factors zᵢ. Each column in Matrix A provides information regarding the underlying application or phenomenon. In one embodiment, Matrix A is indexed by (function of) the PR values. As a result, the entries in Matrix A change for each PR value, which in turn change the optimal solutions for x and scaling factors zᵢ that minimize the cost function in Equation 1.

Next, as shown in block 708, the one or more metrics are computed for each photoplethysmograph model indexed by a PR value by comparing the photoplethysmograph models with a reference photoplethysmograph model. The reference photoplethysmograph model represents the best or a selected photoplethysmograph model for a user associated with the measurement data. In one embodiment, the one or more metrics are associated with the photoplethysmograph model. Example metrics include, but are not limited to, root mean square accuracy (Arms), correlation, L2 norm, L1 norm, Linf norm, power, correlation, and harmonic and morphology analysis matching. Because the one or more metrics are calculated from photoplethysmograph models that are indexed by PR values, the one or more metrics are also indexed by the same PR values.

For example, in some embodiments, the one or more computed metrics are compared with corresponding metrics associated with the reference photoplethysmograph model (reference metric(s)) to determine how close or similar the one or more computed metrics are to the corresponding reference metric(s). Additionally or alternatively, the shape of each photoplethysmograph model is compared with a shape of the reference photoplethysmograph model to determine how similar or dissimilar each photoplethysmograph model is to the reference photoplethysmograph model. In some embodiments, metrics such as the root mean square accuracy (Arms), correlation, L2 norm, L1 norm, Linf norm, correlation, and harmonic and morphology analysis matching can be used to access the degree of compliance (shape similarity) between photoplethysmograph model and reference photoplethysmograph model.

At block 710, an optimal photoplethysmograph model is selected or determined for each wavelength channel and one or more values of interest are estimated or computed. The values of interest can include values of interest for physiological parameter such as SpO2, PR, PI, and/or other physiological parameters of interest. The values of interest are computed by applying classification criteria (algorithms) to the computed metrics (i.e., maximum value, minimum value, a ratio of values, linear and non-linear classification algorithms, etc.). For instance, the best estimate for PR, for given red and infrared data streams, may be obtained by picking the PR value that produces the photoplethysmograph model with the largest normalized power, provided that the corresponding photoplethysmograph model Arms error value (when compared to the most current reference photoplethysmograph model) is less than a specified threshold. The best estimate for SpO2 and PI may be calculated via the scaling factors (red and infrared amplitudes) from the photoplethysmograph model that produced the best estimate for PR.

One or more outliers are then removed from the estimated values of interest to produce a subset of values of interest. In some embodiments, average estimates of the values of interest are produced at block 712. Any suitable technique can be used to remove the outliers.

The subset of values of interest are then provided to a storage device (e.g., memory) and/or an output device (block 714). For example, the one or more values of interest can be displayed on a display. Next, as shown in block 716, the reference photoplethysmograph model is updated based on the subset of values of interest and/or the optimal photoplethysmograph model (e.g., the associated optimization variables). In one embodiment, the reference photoplethysmograph model is updated via an update rule that produces a weighted average of the current reference photoplethysmograph model and the optimal photoplethysmograph model.

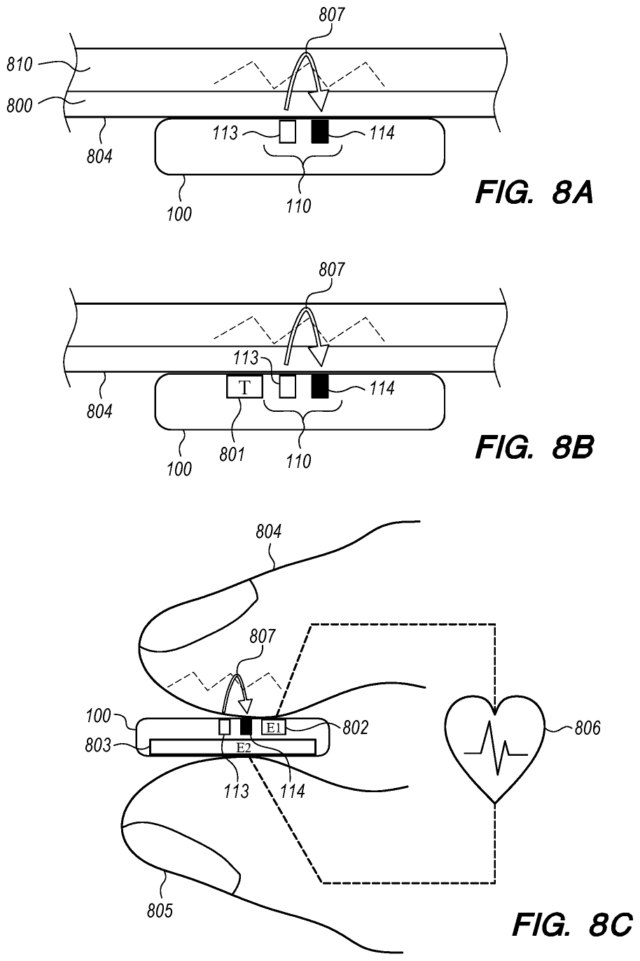

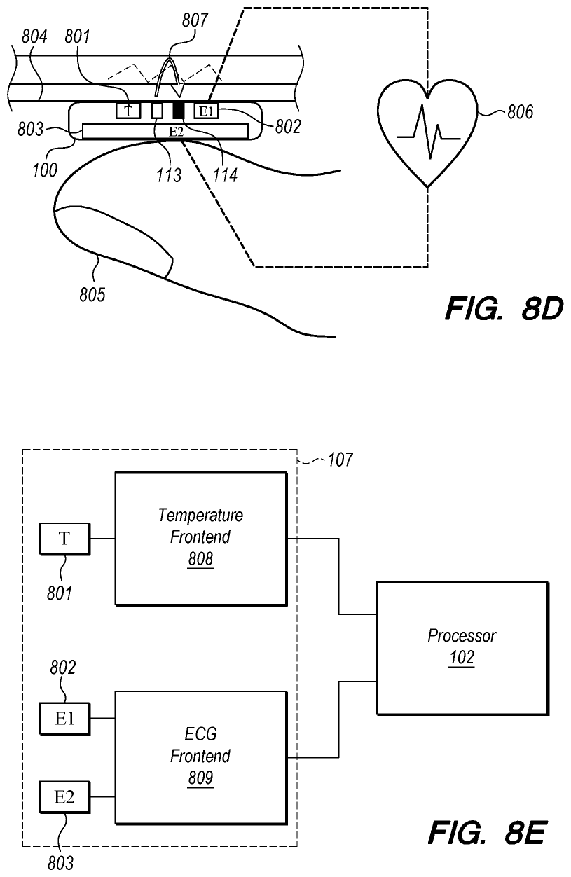

FIGS. 8A-8D depict example embodiments of a low-cost, single-use monitoring device. In FIG. 8A, the monitoring device 100 is affixed to a measurement site 804 and includes the optical sensor 110. As described previously, the optical sensor 110 includes one or more light sources 113 and one or more photodetectors 114. The one or more light sources 113 emit light 807 that penetrates the epidermis 800 and the blood perfused dermis 810 and interacts with the heart's pulsatile signal to create optical pulsatile signals. The optical pulsatile signals (photoplethysmographs) are captured by a frontend circuitry (e.g., instrumentation circuitry 107 and processing device 102 in FIG. 1) connected to the one or more photodetectors 114. The frontend circuitry filters, conditions, and/or converts the optical pulsatile signals to digital signals. These digital signals can be transmitted wirelessly to a host device (e.g., host device 105 using the communication device 103) for further processing and analysis, real-time measurement displaying, warning (alarm) generation, and/or storage of the subject's SpO2, PR, PI, etc.

In FIG. 8B, the monitoring device 100 includes the optical sensor 110 and one or more temperature sensors (represented by temperature sensor (T) 801) in contact with a measurement site 804. The temperature sensor 801 is used to measure core body temperature. For instance, the monitoring device 100 can be attached to the subject's forehead, the ear (posterior auricle), or some other location on the subject's body with superficial temperature correlated to the body's core temperature. The temperature sensor 801 can be connected to temperature frontend circuitry 808 (depicted in FIG. 8E) that conditions, filters, and/or converts the temperature signal to a digital temperature signal. The digital temperature signal can then be transmitted wirelessly to a host device (e.g., host device 105 in FIG. 1) for further processing and analysis, real-time measurement displaying, warning (alarm) generation, and/or storage of the body's core temperature. In one embodiment, the temperature frontend circuitry 808 includes the instrumentation circuitry 107 and the processing device 102 shown in FIG. 1.

The monitoring device 100 in FIG. 8C includes a first electrical contact sensor (El) 802 and a second electrical contact sensor (E2) 803. The example embodiment can be used on measurement sites where either the temperature sensor (T) 801 is not needed, or the superficial temperatures at the measurement sites are not correlated to the body's core temperature. In the illustrated embodiment, the monitoring device 100 is attached to a measurement site 804 (e.g., the subject's digit). The first electrical contact sensor (E1) 802 is positioned on an interior side of a first surface of the monitoring device 100, where the exterior side of the first surface is in contact with the measurement site when the monitoring device is attached to the measurement site. Accordingly, the first electrical contact sensor (E1) 802 is in constant contact (or near constant contact) with the measurement site when the monitoring device 100 is positioned on the measurement site.

The second electrical contact sensor (E2) 803 is positioned on an interior side of a second surface of the monitoring device 100. In FIG. 8C, the second surface is opposite the first surface, although other embodiments are not limited to this configuration. The second electrical contact sensor (E2) can be located on a side of any surface such that the second electrical contact sensor (E2) 803 is not in contact with the measurement site when the monitoring device 100 is attached to the measurement site.

When the monitoring device 100 is placed or attached on the measurement site 804, the subject can touch the electrical contact sensor 803 with a digit 805 of the opposite hand to create a closed electrical path to the subject's heart 806 and enable the measurement of the subject's heart electrical activity (e.g., an electrocardiogram (ECG)). The first and the second electrical contact sensors 802, 803 can be connected to ECG frontend circuitry 809 (depicted in FIG. 8E). The ECG frontend circuitry 809 conditions, filters, and/or converts the ECG signal to an ECG digital signal. The ECG digital signal can be sent wirelessly to a host device (e.g., host device 105 in FIG. 1) for further processing and analysis, real-time displaying of measurements, warning (alarm) generation, and/or storage of the ECG signal. In one embodiment, the ECG frontend circuitry 809 is part of the instrumentation circuitry 107 and includes the processing device 102 shown in FIG. 1.

With reference to FIG. 8D, the example embodiment includes the temperature sensor (T) 801 and the first and the second electrical contact sensors 802 803. In the illustrated embodiment, the measurement site 804 is assumed to have a superficial temperature that is correlated to the body's core temperature (i.e., forehead, ear (posterior auricle), armpit, etc.) or have a temperature that can be mapped to correlate with the body's core temperature (i.e., digit, etc.). Like FIG. 8C, the subject creates a closed path to the subject's heart 806 when the monitoring device 100 is placed on the measurement site 804 such that the first electrical contact sensor (El) 802 contacts the measurement site and the subject touches the second electrical contact sensor (E2) 803 with the digit 805 or any other body part that creates a closed electrical path to the subject's heart 806. The first and the second electrical contact sensors 802, 803 depicted in FIGS. 8C and 8D are replaced with electrical capacitive contact sensors in embodiments where electrical isolation between the monitoring device 100 and the subject's body is preferred. In such embodiments, the ECG frontend circuitry 809 can have a high input impedance to enable the acquisition of heart's ECG without any low-frequency harmonic distortions (attenuation) that can be created by the electrical capacitive contact sensors.

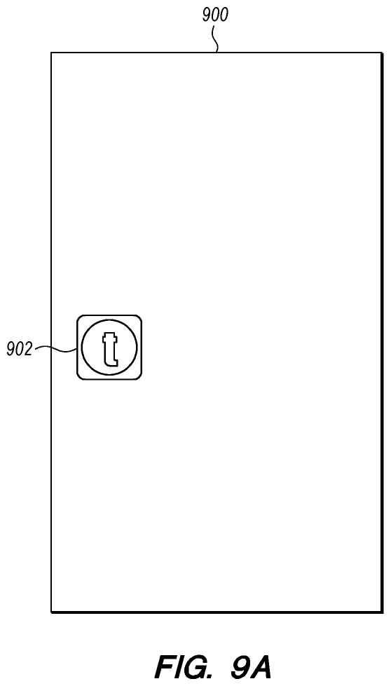

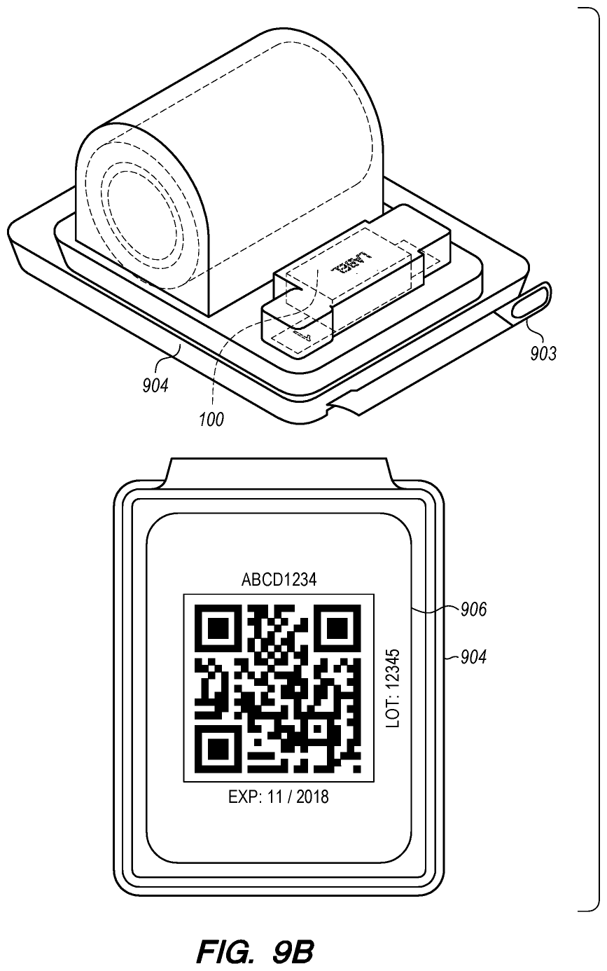

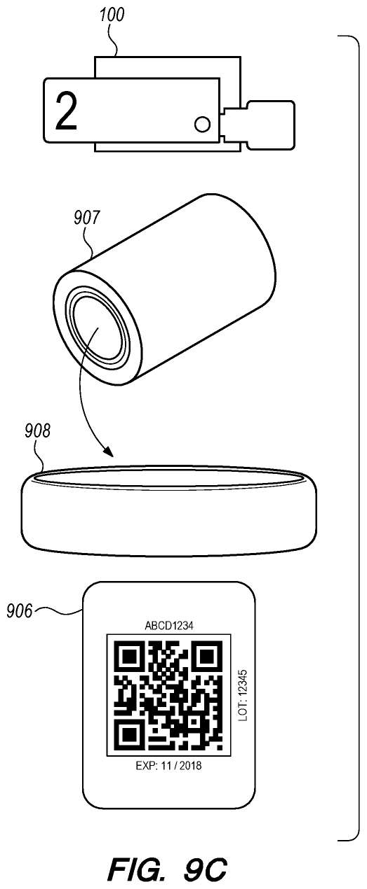

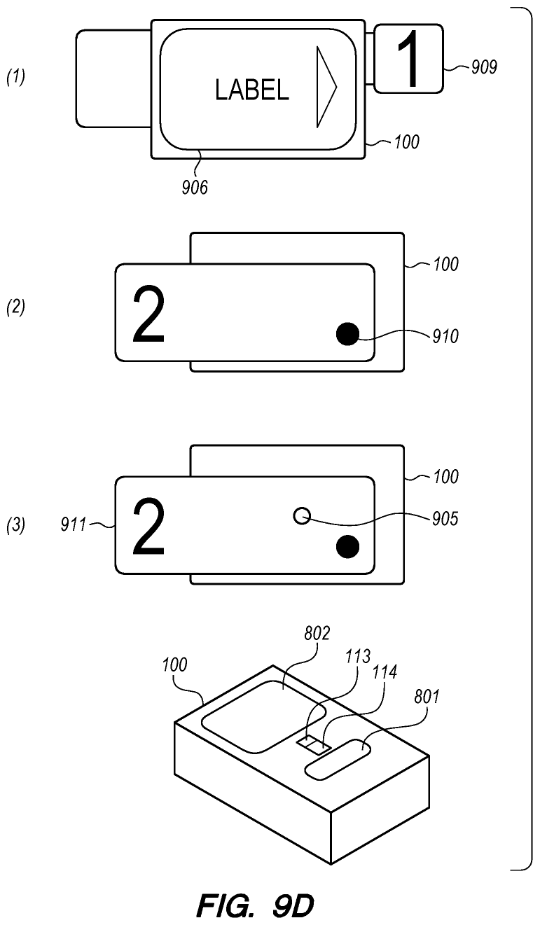

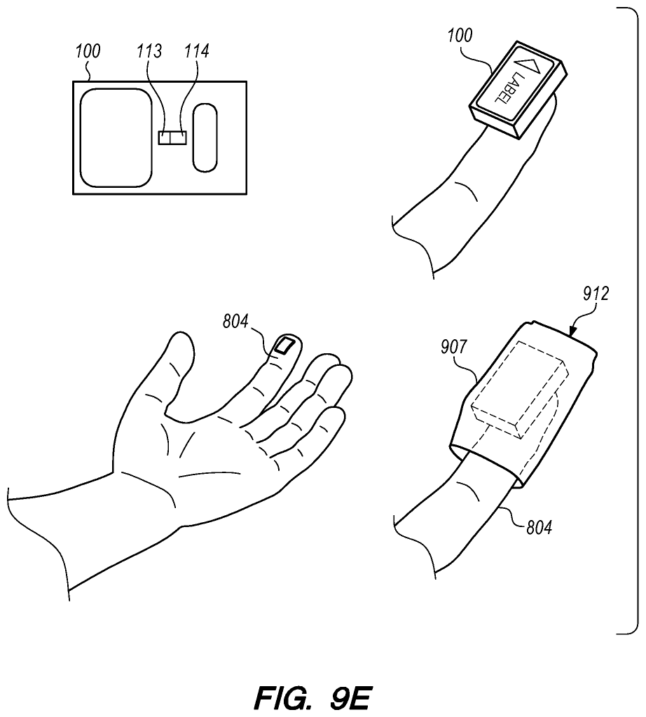

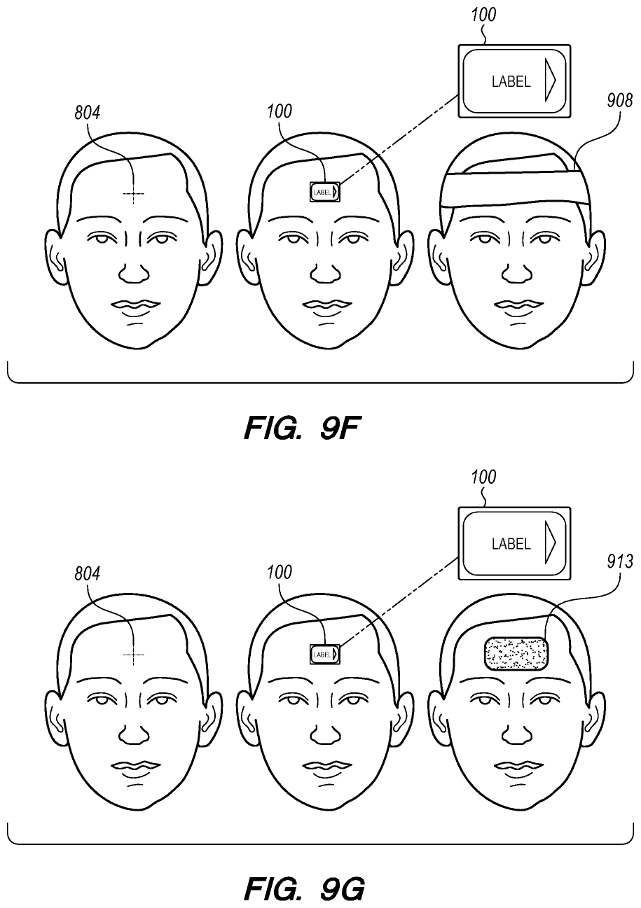

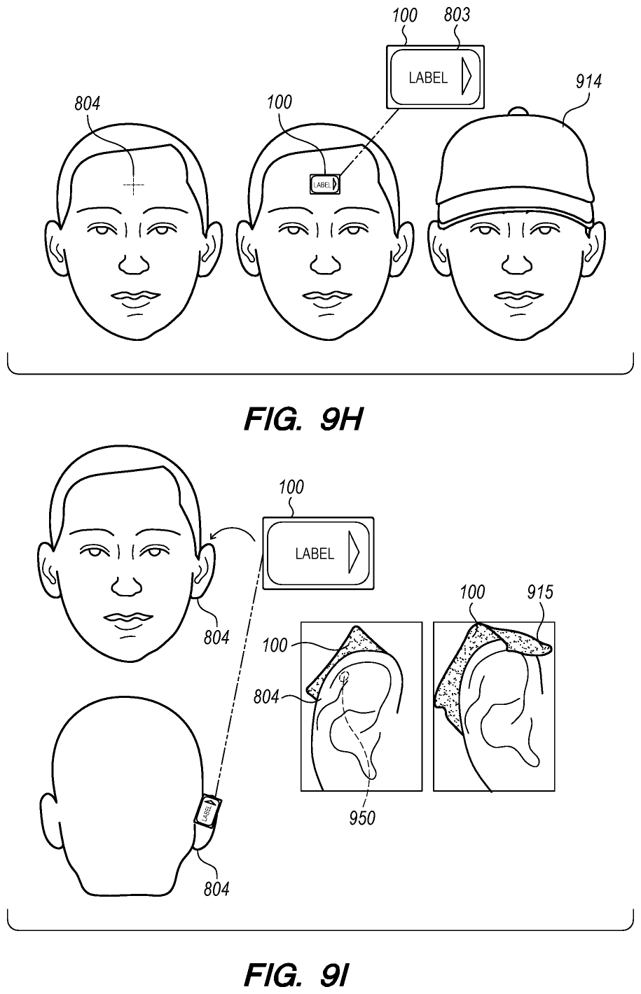

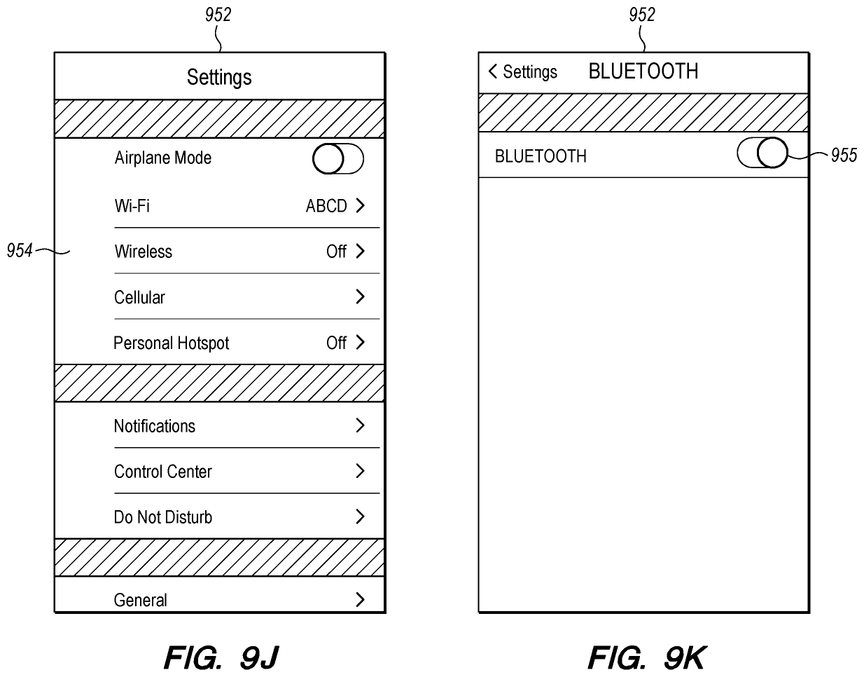

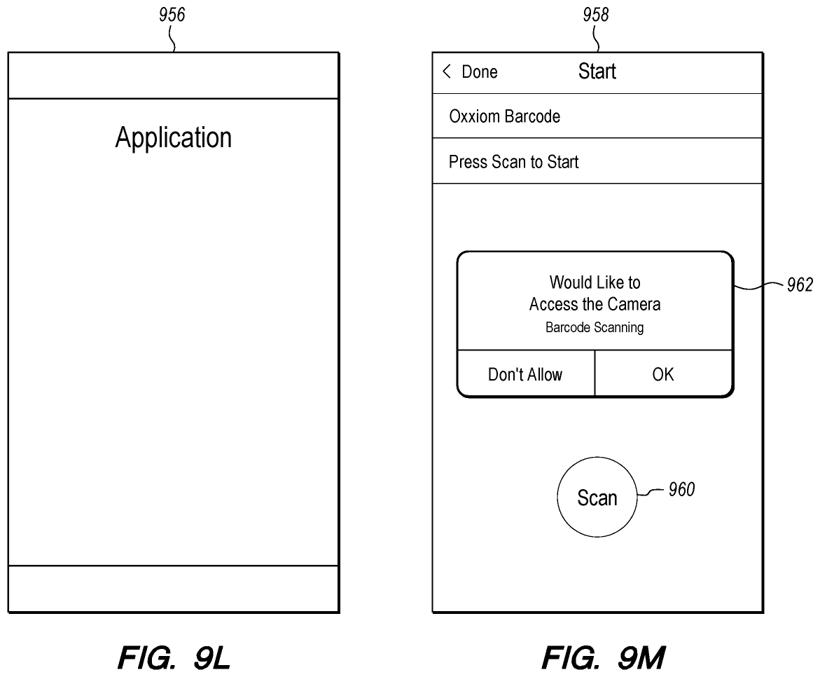

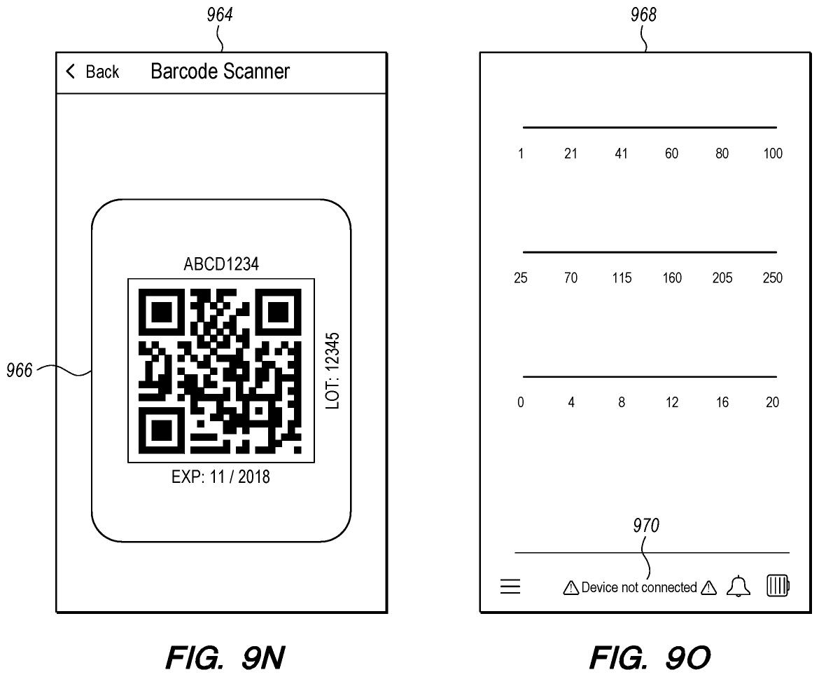

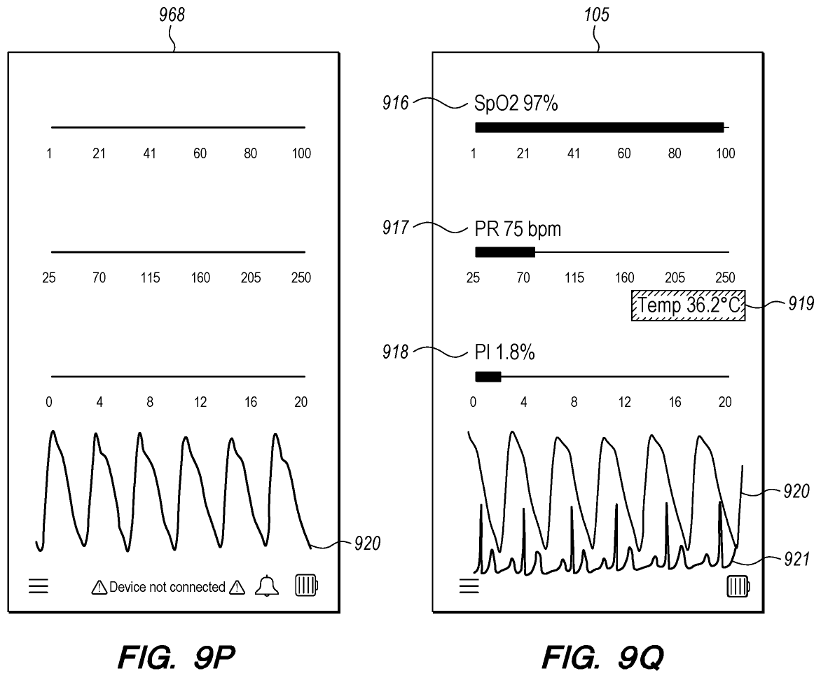

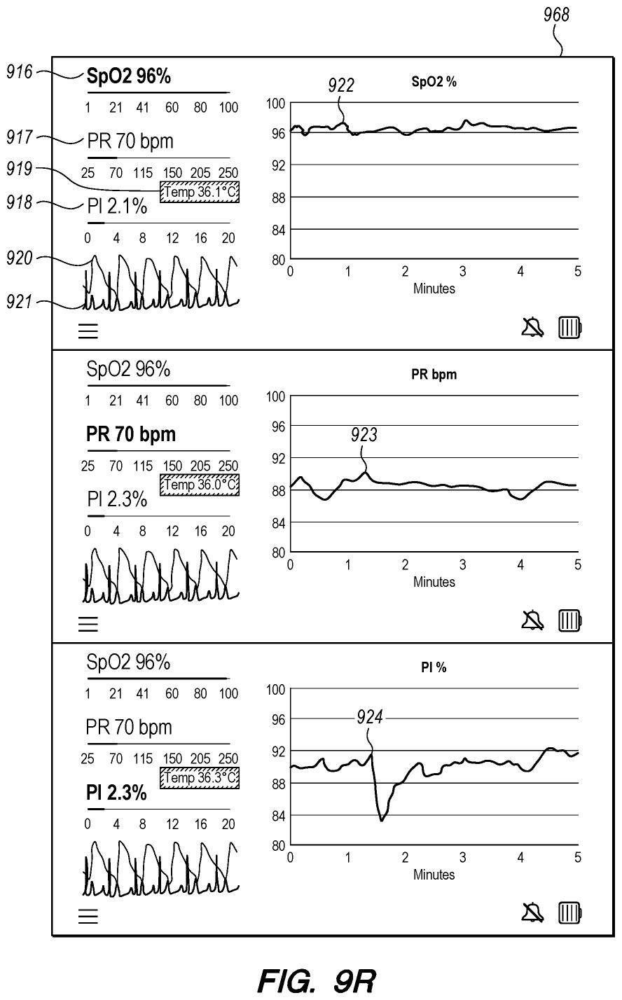

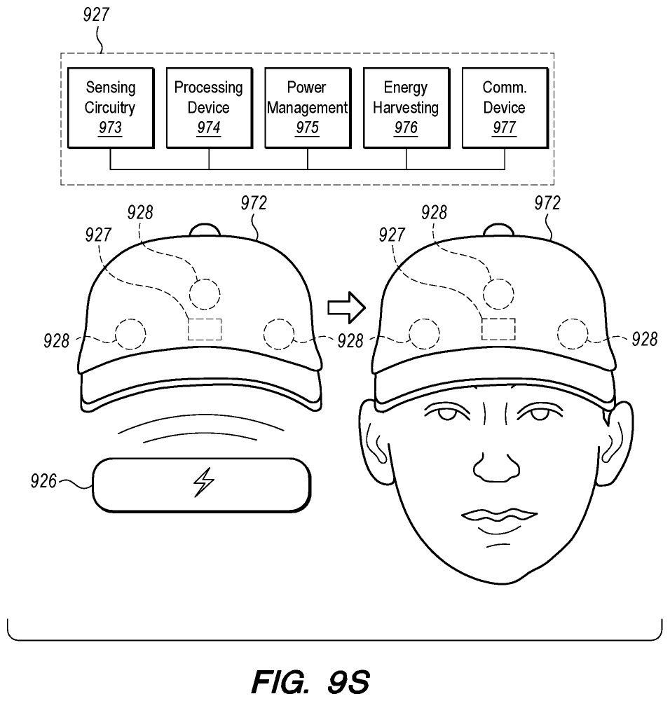

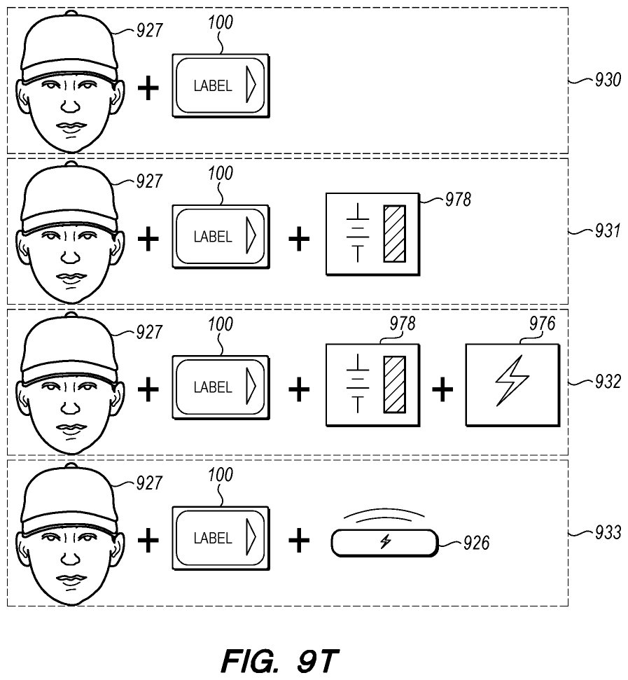

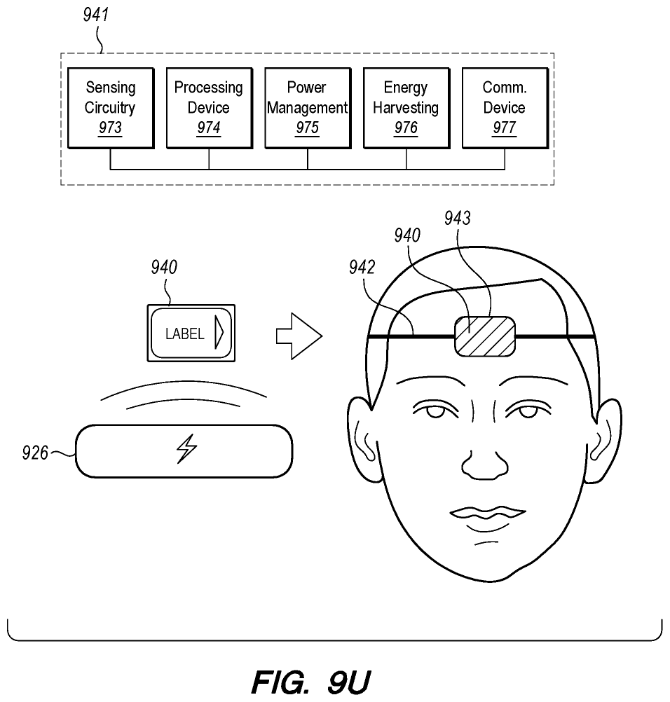

FIG. 9A depicts the application program that works with the monitoring device (hereinafter the “application program”) installed in a host device. In one embodiment, the application program is downloaded from an online application program site or the website of the application program provider and installed in the host device. An icon 902 representing the application program can be displayed on a screen 900 on the host device. For example, the monitoring device is the OXXIOM pulse oximeter and the application program is the OXXIOM Application by True Wearables, Inc. Example host devices include, but are not limited to, a smart watch, a desktop computer, a laptop computer, and a cell phone or other mobile computing device.

FIGS. 9B-9C illustrate the monitoring device and accessories. To prevent tampering with the monitoring device 100 during storage and/or transport, an anti-tamper tab 903 seals the package 904 until it is ready for use by the user. When the user is ready to use the monitoring device 100, the user tears off the anti-tamper tab 903 and removes the product label 906 with the barcode. In FIG. 9C, the monitoring device 100 and the accessories are removed from the package 904. In this example, in addition to the monitoring device 100 and the product label 906, the package 904 contains a roll of tape 907 and a headband 908. In one embodiment, the tape 907 is a self-adhering gentle breathable tape. The headband 908 can be stored inside the roll of tape 907 and removed from the roll of tape 907 when the roll of tape 907 is removed from the package 904) (removal of headband 908 represented by arrow).

FIG. 9D details the steps to activate the monitoring device. In step one, the tab 909 labeled “1” is removed to close an electrical contact and turn on the monitoring device 100. In one embodiment, the tab 909 is made of conductive tape. In step two, an indicator 910 (e.g., the dot) on the exterior surface of the monitor device 100 is pressed by the user to confirm the electrical contact is closed to turn on monitoring device. When the monitoring device 100 is turned on, a light 905 (e.g., a green light) is turned on and indicates the monitoring device 100 is able to be operably connected to the application program on the host device. In one instance, the light 905 can be part of the one or more light sources 113 of the optical sensor to reduce costs and device footprint.

In step three, the tab 911 labeled “2” is removed to expose an adhesive tape used to attach the monitoring device 100 to a measurement site. For example, the adhesive tape can be biocompatible adhesive tape. In one embodiment, the second electrical contact sensor (e.g., 803 in FIG. 8D) is the label 906 depicted in step one of FIG. 9D. The label 906 can be made out of a metallic adhesive tape so as to have the dual function of identifying the monitoring device 100 and functioning as the second electrical contact sensor. Additionally or alternatively, the first electrical contact sensor (e.g., 802 in FIG. 8D) and the temperature sensor (e.g., 801 in FIG. 8D) can be part of the adhesive tape that is exposed when the tab 911 is removed. The adhesive tape, as part of the outer shell or enclosure of the monitoring device, can be designed to have good adherence to the subject's skin, as well as good electrical and thermal properties to enable the accurate measurement of the body's core temperature and the heart's ECG.

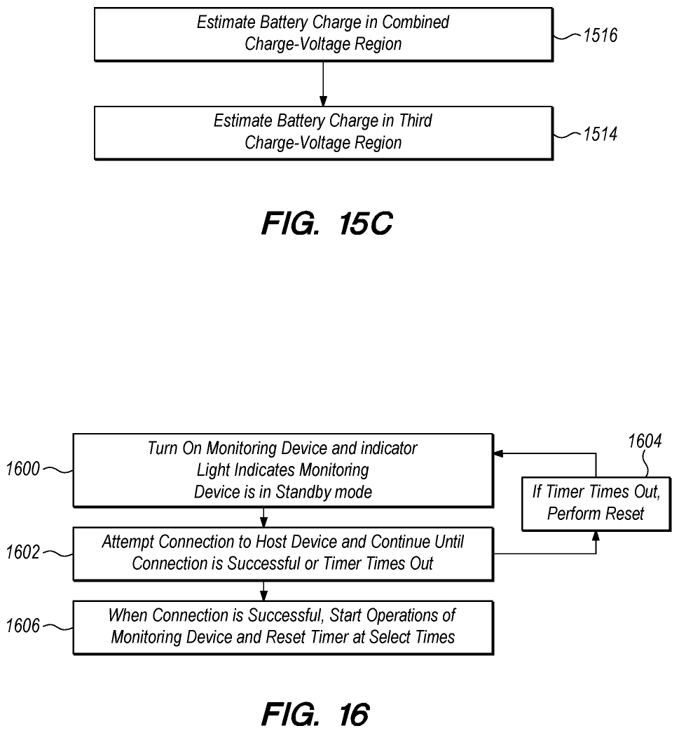

FIGS. 9E-91 depict a workflow showing processes for attaching various example monitoring devices to measurement sites. FIG. 9E shows the process of positioning the monitoring device 100 on the user (e.g., to a digit of the user). In the illustrated embodiment, steps one through three of FIG. 9D have already been performed. The monitoring device 100 is attached to the measurement site 804 (e.g., the fingertip) with the light source(s) 113 and photodetector(s) 114 of the optical sensor (and the temperature sensor 801 and/or the first electrical contact sensor 802 if included in the monitoring device 100) placed on, and in contact with, the measurement site 804. In some embodiments, the monitoring device 100 is wrapped by the tape 907 to improve the optical, electrical, and/or thermal coupling between the monitoring device 100 and the measurement site 804. The tape 907 can also protect the monitoring device 100 against a variety of events, including, but not limited to, mechanical shocks, dislodging, and direct sunlight exposure.

However, in embodiments where the monitoring device 100 includes the first and the second electrical contact sensors (e.g., 802, 803 in FIG. 8D), the tape 907 may cover the second electrical contact sensor (e.g., 803 in FIG. 8D) depending on how the tape 907 is wrapped around the measurement site 804 and the monitoring device 100. Accordingly, conductive fibers (not shown) can be included in the tape 907 to produce a tape with conductive properties. The conductive fibers in the tape 907 create an electrical contact between the second electrical contact sensor and the tape 907. A closed electrical path to the subject's heart (e.g., 806 in FIG. 8D) is created whenever the subject touches the conductive self-adhering tape 907 with another body part (e.g., the opposite hand 805 in FIG. 8D). In some instances, the conductive fibers are arranged so as not to block the wave frequencies in the applicable wireless frequency range (for instance, for Bluetooth Low Energy, the frequency range is between 2400 and 2483.5 MHz) to enable the monitoring device 100 to communicate wirelessly with the host device.

In another embodiment, the tape 907 with the conductive fibers can be made part of the monitoring device 100 so as to perform the functions of boosting the gain of the wireless antenna in the monitoring device 100. To boost the wireless efficiency, when using the conductive tape 907 with the conductive fibers, the wrapping of the tape 907 around the measurement site 804 can be done in a way to leave a cavity (or aperture, gap) 912 at the fingertip extremity. A cavity 912 can be created based on how the tape 907 is wrapped around the monitoring device 100 and the measurement site 804 (e.g., the fingertip) and/or by the way the tape 907 is designed and/or attached to the monitoring device 100.

The conductive fibers in the tape 907 can improve the optical, electrical and/or thermal coupling (contact) with the measurement site 804 when pressure is applied to the monitoring device 100 at the measurement site 804. Additionally or alternatively, the conductive fibers may serve as the second electrical contact sensor (e.g., 803 in FIG. 8D) that enables the user (subject) to create a closed electrical path to the heart when the subject touches the tape 907 with the conductive fibers.

FIG. 9F depicts the monitoring device positioned on the subject's forehead. In the illustrated embodiment, steps one through three of FIG. 9D have already been performed. The monitoring device 100 is attached to the measurement site 804 (e.g., the forehead) with the light source(s) 113 and photodetector(s) 114 of the optical sensor (and the temperature sensor 801 and/or the first electrical contact sensor 802 if included in the monitoring device 100) placed on, and in contact with, the measurement site 804.

In some instances, the use of the forehead as a measurement site 804 can result in improved measurements for SpO2, PR and PI. The perfused tissue in the forehead region is a few millimeters thick and is suitable for a reflective pulse oximeter that has a relatively small separation (gap) between the one or more light sources and the one or more photodetectors (e.g., light source(s) 113 and photodetector(s) 114 in FIG. 8A). In some embodiments, the monitoring device 100 has a separation gap between 2.5 mm and 7 mm. However, depending on the optical sensor design, separation gaps smaller than 2.5 mm can be attained. The monitoring device 100 can probe the shallow and perfused blood tissue from the forehead, providing waveforms with relatively high signal-to-noise ratio, and enabling the accurate detection of SpO2, PR, and PI, even at very low perfusion levels.

The forehead as a measurement site 804 may also be advantageous during physical activity. The measurement site 804 is less prone to motion artifacts given that it is located on the subject's head. As a mechanism of self-protection against brain injuries, the body's motion effects are always minimized at the subject's head, through motion dumping enabled by the skeleton and muscles. In addition, the forehead is subjected to shorter transport delays to physiological changes (i.e., SpO2, PR, and/or PI changes. etc.), and also shows less vasoconstrictor response to cold, given its proximity to the brain. Optionally, a headband 908 (see FIG. 9C) can be used to protect the monitoring device 100 against various events, such as mechanical shocks, dislodging, and direct sunlight exposure. The headband 908 can also improve optical, thermal and/or electrical coupling with the measurement site 804 (e.g., the forehead).

However, in embodiments where the monitoring device 100 includes the first and the second electrical contact sensors (e.g., 802, 803 in FIG. 8D), the headband 908 may cover the second electrical contact sensor (e.g., 803 in FIG. 8D). Accordingly, conductive fibers (not shown) can be included in the headband 908 to produce a fabric or headband with conductive properties. The conductive fibers in the headband 908 create an electrical contact between the second electrical contact sensor and the headband 908. A closed electrical path to the subject's heart (e.g., 806 in FIG. 8D) is created whenever the subject touches the headband 908 with the conductive fibers with a body part (i.e., a fingertip 805 in FIG. 8D).

To enable the monitoring device 100 to communicate wirelessly with the host device (e.g., 105 in FIG. 1), the conductive fibers in the headband 908 can be arranged so as not to block the wave frequencies in the applicable wireless frequency range. In another embodiment, the headband 908 with the conductive fibers can be made part of the monitoring device 100 so as to perform the functions of boosting the gain of the wireless antenna in the monitoring device 100 through the conductive fibers, and/or improving the optical, electrical, and thermal coupling (contact) with the measurement site 804 (e.g., forehead) when pressure is applied to the monitoring device 100 at the measurement site 804. Additionally or alternatively, the conductive fibers may serve as the second electrical contact sensor (e.g., 803 in FIG. 8D) that enables the user (subject) to create a closed electrical path to the heart whenever the subject touches the headband 908 with conductive fibers using a body part (i.e., fingertip 805 in FIG. 8D).

FIG. 9G depicts the monitoring device 100 placed on the subject's forehead with the light source(s) 113 and photodetector(s) 114 of the optical sensor (and the temperature sensor 801 and/or the first electrical contact sensor 802 if included in the monitoring device 100) placed on, and in contact with, the measurement site 804. In this embodiment, an adhesive bandage 913 is used to protect the monitoring device 100 against various events, including mechanical shocks, dislodging, and direct sunlight exposure. The adhesive bandage 913 can also improve the optical, thermal and/or electrical coupling with the measurement site 804 (e.g., forehead).

However, in embodiments where the monitoring device 100 includes the first and the second electrical contact sensors (e.g., 802, 803 in FIG. 8D), the adhesive bandage 913 may cover the second electrical contact sensor (e.g., 803 in FIG. 8D). Accordingly, conductive fibers (not shown) can be included in the adhesive bandage 913 to produce a bandage with conductive properties. The conductive fibers in the bandage 913 create an electrical contact between the second electrical contact sensor (e.g., 803 in FIG. 8D) and the bandage 913. A closed electrical path to the subject's heart (e.g., 806 in FIG. 8D) is created whenever the subject touches the bandage 913 with the conductive fibers with a body part (i.e., a fingertip 805 in FIG. 8D).

To enable the monitoring device 100 to communicate wirelessly with a host device (e.g., 105 in FIG. 1), the conductive fibers in the bandage 913 can be arranged so as not to block wave frequencies in the applicable wireless frequency range. In another embodiment, the bandage 913 with the conductive fibers can be made part of the monitoring device 100 so as to perform the functions of boosting the gain of the wireless antenna in the monitoring device 100 through the conductive fibers, and/or improving the optical, electrical, and thermal coupling (contact) with the measurement site 804 (e.g., forehead) when pressure is applied to the monitoring device 100 at the measurement site 804. Additionally or alternatively, the conductive fibers may serve as the second electrical contact sensor (e.g., 803 in FIG. 8D) that enables the user (subject) to create a closed electrical path to the heart whenever the subject touches the headband 908 with conductive fibers using a body part (i.e., fingertip 805 in FIG. 8D).

FIG. 9H depicts the monitoring device 100 placed on the subject's forehead. The monitoring device 100 is attached to the measurement site 804 (e.g., the forehead) with the light source(s) 113 and photodetector(s) 114 of the optical sensor (and the temperature sensor 801 and/or the first electrical contact sensor 802 if included in the monitoring device 100) placed on, and in contact with, the measurement site 804. In this embodiment, a hat 914 can be used to protect the monitoring device 100 against various events, including mechanical shocks, dislodging, and direct sunlight exposure. The hat 914 can also improve optical, thermal and/or electrical coupling with the measurement site 804 (e.g., the forehead).

However, in embodiments where the monitoring device 100 includes the first and the second electrical contact sensors (e.g., 802, 803 in FIG. 8D), the hat 914 may cover the second electrical contact sensor (e.g., 803 in FIG. 8D). Accordingly, conductive fibers (not shown) can be included in the hat 914 to produce a fabric or a hat 914 with conductive properties. The conductive fibers in the hat 914 create an electrical contact between the second electrical contact sensor and the hat 914. A closed electrical path to the subject's heart (e.g., 806 in FIG. 8D) is created whenever the subject touches the hat 914 with the conductive fibers with a body part (i.e., a fingertip 805 in FIG. 8D).

To enable the monitoring device 100 to communicate wirelessly with a host device (e.g., 105 in FIG. 1), the conductive fibers in the hat 914 can be arranged so as not to block wave frequencies in the applicable wireless frequency range. In another embodiment, the hat 914 with the conductive fibers can be made part of the monitoring device 100 so as to perform the functions of boosting the gain of the wireless antenna in the monitoring device 100 through the conductive fibers, and/or improving the optical, electrical, and thermal coupling (contact) with the measurement site 804 (e.g., forehead) when pressure is applied to the monitoring device 100 at the measurement site 804. Additionally or alternatively, the conductive fibers may serve as the second electrical contact sensor (e.g., 803 in FIG. 8D) that enables the user (subject) to create a closed electrical path to the heart whenever the subject touches the hat 914 with conductive fibers using a body part (i.e., fingertip 805 in FIG. 8D).

FIG. 91 depicts the monitoring device 100 placed on the subject's posterior auricle (ear). The monitoring device 100 is attached to the measurement site 804 (e.g., the ear) with the light source(s) 113 and photodetector(s) 114 of the optical sensor (and the temperature sensor 801 and/or the first electrical contact sensor 802 if included in the monitoring device 100) placed on, and in contact with, the measurement site 804. In some instances, depending on the intensity of the light and the skin color of the user, the light emitted by the one or more light source(s) 113 may be visible through the ear's cartilage (see region 950).

In one aspect, the use of the posterior auricle as a measurement site 804 can result in improved measurements for SpO2, PR and PI. The thickness of the ear cartilage in this region of the ear is typically only a few millimeters thick. Thus, the measurement site 804 is suitable for a reflective pulse oximeter that has a small separation (gap) between the one or more light sources and the one or more photodetectors of the optical sensor (e.g., 113, 114 in FIG. 8D).

The measurement site 804 enables the monitoring device 100 to probe the shallow and perfused blood tissue from the posterior auricle, providing waveforms with high signal-to-noise ratio and enabling the accurate detection of SpO2, PR, and PI, even at very low perfusion levels. The posterior auricle as a measurement site 804 is also advantageous during physical activity. The measurement site 804 (e.g., the ear) is less prone to motion artifacts given that it is located on the subject's head. As a mechanism of self-protection against brain injuries, the body's motion effects are always minimized at the subject's head through motion dumping enabled by the skeleton and muscles. In addition, the posterior auricle is subjected to shorter transport delays to physiological changes (i.e., SpO2, PR, and/or PI changes. etc.), and also shows less vasoconstrictor response to cold, given its proximity to the brain.

In some embodiments, an adhesive tape 915 can be used to protect the monitoring device 100 against various events, including mechanical shocks, dislodging, and direct sunlight exposure. The adhesive tape 915 may also improve optical, thermal and/or electrical coupling with the measurement site 804 (posterior auricle). However, in embodiments where the monitoring device 100 includes the first and the second electrical contact sensors (e.g., 802, 803 in FIG. 8D), the adhesive tape 915 may cover the second electrical contact sensor (e.g., 803 in FIG. 8D). Accordingly, conductive fibers (not shown) can be included in the adhesive tape 915 to produce an adhesive tape 915 with conductive properties. The conductive fibers in the adhesive tape 915 create an electrical contact between the second electrical contact sensor and the adhesive tape 915. A closed electrical path to the subject's heart (e.g., 806 in FIG. 8D) is created whenever the subject touches the adhesive tape 915 with the conductive fibers with a body part (i.e., a fingertip 805 in FIG. 8D).

To enable the monitoring device 100 to communicate wirelessly with a host device (e.g., 105 in FIG. 1), the conductive fibers in the adhesive tape 915 can be arranged so as not to block wave frequencies in the applicable wireless frequency range. In another embodiment, the adhesive tape 915 with the conductive fibers can be made part of the monitoring device 100 so as to perform the functions of boosting the gain of the wireless antenna in the monitoring device 100 through the conductive fibers, and/or improving the optical, electrical, and thermal coupling (contact) with the measurement site 804 (e.g., ear) when pressure is applied to the monitoring device 100 at the measurement site 804. Additionally or alternatively, the conductive fibers may serve as the second electrical contact sensor (e.g., 803 in FIG. 8D) that enables the user (subject) to create a closed electrical path to the heart whenever the subject touches the adhesive tape 915 with conductive fibers using a body part (i.e., fingertip 805 in FIG. 8D).

FIGS. 9J-9P illustrate the steps to start the application program in a host device, connect the monitoring device to the host device, and begin data exchange between the monitoring device and the host device to produce measurements and waveforms. As shown in FIGS. 9J and 9K, a wireless communication device in the host device (e.g., 116 in FIG. 1) is enabled via a setting 954 in the settings user interface 952 on the host device. For example, in one embodiment, the BLUETOOTH wireless communication device is enabled using switch 955 (see FIG. 9K).

Next, as shown in FIG. 9L, the user launches the application program that interacts with the monitoring device, which can optionally cause a startup screen 956 with information about the application program to be displayed (e.g., information such as the application program name, manufacturer's name, copyright, etc.). A screen 958 with a selectable element 960 is displayed (FIG. 9M). In FIG. 9M, the selectable element 960 is a button labeled “scan”, although other embodiments are not limited to this implementation. When the user launches the application program for the first time (e.g., the application program has not been used before), in some embodiments the application program is locked into the screen 958 until the user scans a valid barcode from a product label (e.g., product label 906 in FIG. 9B).

When the user selects the selectable element 960 (e.g., the scan button), an optional notification 962 may be presented stating the application program is requesting access to an imaging device (e.g., the camera) on the host device (FIG. 9M). The imaging device is used to scan the barcode on the product label. FIG. 9N illustrates an example screen 964 displaying an image 966 of the scanned product label. In some embodiments, an indicator (e.g., a colored square; not shown) is produced on the screen of the host device around the image 966 of the scanned barcode. Additionally or alternatively, an audio sound, such as a beep, is produced to indicate the barcode has been scanned and is valid. If the scanned barcode is not valid, then a different indicator or an indicator having a different color (e.g., a red square) is displayed adjacent to or around the invalid barcode. In some instances, a different audio sound (e.g., a bell) is produced in addition to, or as an alternative to, the indicator. The host device produces haptic feedback (e.g., vibrations produced by a haptic transducer) to indicate the barcode is invalid in other embodiments.

The application program can display (or cause to be displayed) a measurement screen 968 that includes a notification 970 that the monitoring device is not connected to the application program (FIG. 9P). The measurement screen 968 can be presented in a portrait mode (see FIG. 9P) or in a landscape mode. As shown in FIG. 9P, a waveform chart 920 (photoplethysmograph) is displayed in the measurement screen 968.

In FIG. 9Q, the SpO2 916, PR 917, and PI 918 measurement gauges, the body core temperature 919 gauge, and the waveform chart 920 (photoplethysmographs) are displayed in the measurement screen 968. When the monitoring device includes the first and the second electrical contact sensors (e.g., 802, 803 in FIG. 8D), and the user touches the second electrical contact sensor with a body part (e.g., a finger), a spot-check ECG waveform 921 is displayed in the measurement screen 968. The spot-check ECG waveform 921 may be updated in real-time on the measurement screen 968 until the body part (e.g., the finger) is not in contact with the second electrical contact sensor. When the user removes the body part from the second electrical contact sensor, the ECG waveform 921 may be frozen on the measurement screen 968, and/or may disappear from the measurement screen 968. In some embodiments, the user can set a setting for the ECG waveform 921 to remain frozen or to be removed.

FIG. 9R depicts the measurement screen 968 displaying SpO2 922, PR 923, and PI 924 trends in addition to the SpO2 916, PR 917, and PI 918 measurement gauges, the body core temperature 919 gauge, the waveform chart 920, and the ECG waveform 921. Fewer gauges and/or waveforms can be presented in the measurement screen 968 in other embodiments. In some embodiments, the user can select which of the SpO2 922, PR 923, and PI 924 trends to display on the measurement screen 968. For example, the user can touch a particular gauge on the measurement screen 968 to display a corresponding trend.

FIG. 9S depicts an example embodiment of a wearable hat that includes a monitoring device. The wearable hat 972 includes built-in hat circuitry 927 that turns the hat 972 into a wearable device for medical, fitness, and/or wellness applications. The hat 972 can further include built-in electroencephalogram (EEG) electrodes 928. The EEG electrodes 928 can be part of the fabric of the hat 972 or attached to the hat 972, depending on the application. Other optical, temperature, electrical contact, electrical capacitive contact, ultrasound, etc. sensors can be part of the hat circuitry 927 or can be distributed around the hat layout (as the EEG electrodes) and/or part of the hat's fabric, depending on the application.

The hat circuitry 927 may or may not include some or all of the circuitry in the monitoring device (e.g., monitoring device 100 in FIG. 1). The hat circuitry 927 senses one or more physiological measurements and/or environmental data, processes and conditions the physiological and/or environmental data, and sends the data wirelessly to a host device (e.g., 105 in FIG. 1). The data is transmitted to the host device in real time in one embodiment. For example, the signals from the photodetector(s), the EEG electrodes, and/or the temperature sensor are transmitted to the host device.

The host device processes and analyzes the data, displays one or more measurements and trends, generates an alarm or warning, stores the data, shares the data, and the like. In one embodiment, the hat circuitry 927 has the following functionalities:
1. Sensing Circuitry—The hat circuitry 927 includes sensing circuitry 973 such as electronics and sensors, as discrete and/or distributed components, standard integrated circuits, and/or Application-Specific Integrated Circuit (ASIC), to sense continuously or at select times the body temperature, environmental temperature, environmental pressure, environmental ultraviolet (A, B, and/or C bands), body hydration, non-invasive blood total hemoglobin, carboxyhemoglobin and methemoglobin, SpO2, PR, PI, plethysmographs, non-invasive blood glucose levels, respiration rate, user's activity and calorie consumption, EEG, etc., and spot-check measurements of ECG waveforms.
2. Processing Device—A processing device 974, such as a low-power processing device (e.g., ARM-based, ASIC, etc.) receives the data from the sensing circuitry 973, processes the data, and transmits the data wirelessly to a host device (e.g., 105 in FIG. 1) using the communication device 977. In some embodiments, a processing device, such as ASIC circuitry, performs specialized signal processing functions to reduce overall power consumption and the footprint of the hat circuitry 927. In a non-limiting example, the processing device 974 performs functions having a low complexity and low latency (i.e., hard real-time processing) and the host device processes data having a higher complexity and latency (i.e., soft real-time processing). The processing device 974 can be part of the hat circuitry 927 or can be distributed around the hat layout and/or part of the hat's fabric, depending on the application.
3. Power Management Circuitry—The power management circuitry 975 includes a battery and manages the voltages and loads, battery life, and charging of the battery. In one embodiment, the battery in the hat 972 is charged through a wireless charger 926. The wireless charger 926 minimizes circuitry isolation requirements and eliminates the need for cables and/or connectors. It may also be more convenient for the user given that to charge the battery in the hat 972, the user can place the hat on top or near the wireless charger 926. The voltages can be regulated through boost and buck converters that can be designed to provide voltage levels with maximum noise levels that match the requirements of the battery. The power management unit can be part of the hat circuitry 927 or can be distributed around the hat layout and/or part of the hat's fabric, depending on the application.
4. Energy Harvesting Circuitry—The energy harvesting circuitry 976 can be composed of pyroelectric, piezoelectric, thermoelectric, photovoltaic, ambient-radiation, transducers and electronics that convert some or all forms of energy in the environment into electric power that can power the hart circuitry 927 directly, or that can be used to recharge the battery in the power management circuitry 975 to recharge or increase the battery life. The transducers and electronics can be part of the hat circuitry 927 or can be distributed around the hat layout and/or part of the hat's fabric, depending on the application.
5. Communication Device—Any suitable wireless communication device 977 can be used. In one embodiment, the communication device 977 is a wireless radio unit for receiving and sending data to a host device (e.g., 105 in FIG. 1) or to a network (e.g., 120 in FIG. 1). In a non-limiting example, the hat can communicate wirelessly with a router using IPV6 (Internet protocol Version 6) over the BLUETOOTH Smart protocol. In one embodiment, the communication device 977 supports multiple low-energy protocols such as BLUETOOTH Low Energy, ZIGBEE, ANT, or some custom/proprietary low-energy protocol. The communication device 977, including one or more antennas, can be part of the hat circuitry 927, or can be distributed around the hat layout and/or part of the hat's fabric, depending on the application. In particular, the low-energy radio's antenna design can greatly benefit from the hat's layout, materials, and area, so as to enable distributed antenna layouts with very high intrinsic gains in the frequency bands of interest, thereby reducing significantly the radio's power consumption.

FIG. 9T shows several possible configurations for the wearable hat shown in FIG. 9S. Configuration 930 includes the hat 972 and the monitoring device 100. Configuration 930 can be used on single-use disposable applications, where the hat 972 and the hat circuitry 927 are disposed of after the battery is empty of charge. In this configuration 930, the battery is non-rechargeable and as a result, the battery requires no power management circuitry 975 or energy harvesting circuitry 976. In one embodiment, the non-rechargeable battery can be part of the monitoring device 100.

Configuration 931 includes the hat 972, the monitoring device 100, and a removable battery 978. Configuration 931 can be used on multi-use disposable applications, where the hat 972 and the hat circuitry 927 are used multiple times. In this embodiment, whenever the amount of charge on the battery 978 is low or empty, the battery 978 is replaced with a new battery. The battery 978 can be composed of standard non-rechargeable battery (batteries) or can be a custom module with an anti-temper memory that prevents the user from using a battery that is not provided by the manufacturer of the hat 972.

Configuration 932 is similar to the configuration 931, with the addition of the energy harvesting circuitry 976. The energy harvesting circuitry 976 can be used to increase the amount of charge on the removable battery 978 and extend the amount of time the removable battery 978 is used before replacement or recharging.

Configuration 933 includes the hat 926, the monitoring device 100, and a charger (e.g., wireless charger 926). Configuration 933 is used in reusable applications where the battery is rechargeable, and the charger (e.g., the wireless charger 926) is used to charge the battery. In one embodiment, the rechargeable battery, fuel gauge, protection, and wireless charging receiver circuitries can be part of the monitoring device 100.

FIG. 9U depicts an example embodiment of a wearable patch 940. The patch 940 includes the monitoring device 100 and patch circuitry 941 that senses one or more physiological and/or environmental data, processes and conditions the physiological and/or environmental data, and sends the data wirelessly to a host device (e.g., 105 in FIG. 1). In one embodiment, the data is transmitted to the host device in real time.

The host device processes and analyzes the data, displays one or more measurements and/or trends, generates one or more alarms or warnings, stores the data, and/or shares the data with another computing device. In one embodiment, the patch circuitry 941 has similar functionalities as the hat circuitry 927 shown in FIG. 9S. However, given the relatively small size of the patch (when compared to the hat), the patch circuitry 941 is integrated and lightweight.

The patch 940 can be a disposable, single-use patch, a multi-use patch, or a reusable patch. A monofilament 942 can be used to attach the patch to the measurement site (e.g., the forehead) instead of a headband or an adhesive tape. In one embodiment, the monofilament 942 is a transparent or semi-transparent monofilament. The monofilament 942 can be attached to a case 943 with a cavity that fits the patch 940. The case 943 can be made to match the subject's skin color to create a discreet monitoring solution that works on the measurement site (e.g., the subject's forehead). The case 943 with the monofilament 942 can make the monitoring solution more discreet and improve the optical, electrical, and/or thermal coupling between the patch 940 and the measurement site. The monofilament 942 may protect the patch 940 against various events, such as mechanical shocks, dislodging, and direct sunlight exposure.

In a reusable embodiment, the battery in the patch 940 can be a rechargeable battery that is charged through a charger (e.g., a wireless charger 926). The wireless charger 926 can reduce or minimize the circuitry isolation requirements and reduce or eliminate the need for cables and/or connectors. Wireless charging can also be more convenient for the user given that to charge the patch's battery, the user places the patch 940 on top or near the wireless charger 926. The monitoring solution with the patch 940, the case 943, and the monofilament 942 can be applied to measurement sites other than the forehead. For example, measurement sites such as the arm, the leg, the wrist, the foot, the chest, and the neck, can be used. In addition, in some applications, the reusable monitoring solution with the patch 940, the case 943, and the monofilament 942 can be integrated into a single device to reduce costs, and simplify usage workflow to make it more convenient for daily use.

FIGS. 10A-10B depict a workflow of a host device sharing the data received from the monitoring device. FIG. 10 illustrates a screen 1000 that is displayed by the application program on the host device (e.g., host device 105 in FIG. 1). In one embodiment, the application program can share reports 1001, trends 1002, and waveforms 1003 with another computing device based on a selection of the selectable element 1023. In FIG. 10A, the selectable element 1023 is a button labeled “Share”, although other embodiments are not limited to this implementation.

Reports 1001 can be a combination of analyses and charts shared as a file (i.e., PDF, PS, HTML, etc.) or designed to be interactive with the user. Trends 1002 can be a spreadsheet or database (interactive or shared as a file) with data trend measurements over time. Waveforms 1003 can be a database or spreadsheet file that logs the waveforms collected, or the raw data from where the waveforms and other variables of interest can be extracted.

In FIG. 10B, a screen 1024 depicts example data sharing methods, such as Transfer, Wi-Fi, Message, Mail, Cloud, Print, etc. One example of a Transfer data sharing method is AIRDROP by Apple. Available data sharing methods may vary depending on the configuration of the host device 105 and/or the type of data being shared.

In one embodiment, after the monitoring device is connected to the host device and the host device is displaying one or more measurement gauges, one or more waveforms and/or one or more trends (e.g., SpO2, PR, PI, and temperature measurements, photoplethysmograph and/or ECG waveforms) received over a given period of time, the user can share the Reports. FIGS. 10C-10L illustrate example reports with analyses from data collected over a given period of time (e.g., overnight or 569 minutes) by a user wearing the monitoring device 100. The example reports including particular values, and other embodiments can present different values. The example reports are:
1. SpO2 Measurements Over Time—The report 1004 in FIG. 10C shows an example of a SpO2 trend. The report 1004 can be created by the application program on the host device. Normal SpO2 levels are typically between 94% and 100%. However, during sleep, exercise, or situations of high stress, or in high altitude places, the SpO2 readings may reach lower values (i.e., lower than 94%). The example chart 1004 includes an optional footer 1025 with information about the data collected (i.e., identification 1026 of monitoring device used in the data collection, period of data collection 1027, and data and time the report was created 1027). Although not shown in FIGS. 10D-10L, the embodiments shown in FIGS. 10D-10L can also include the footer.
2. PR Measurements Over Time—The report 1005 in FIG. 10D shows an example of PR trend. The report 1005 can be created by the application program on the host device. Normal resting PR for adults typically ranges from 60 to 100 bpm. However, during sleep, exercise, or situations of high stress, or in high altitude places, PR readings may reach higher values. A report having different values can be produced in other embodiments.
3. PI Measurements Over Time—The report 1006 in FIG. 10E shows an example of PI trend. The example report 1006 can be created by the application program on the host device. PI values typically range from 0.02% (very weak pulsatile signal) to 20% (very strong pulsatile signal). Extreme PI values may indicate a situation of discomfort (e.g., cold, hot, stress, etc.). A report having different values can be produced in other embodiments.
4. SpO2 Distribution—The report 1007 in FIG. 10F shows an example of SpO2 distribution. The example report 1007 can be created by the application program on the host device. As shown in the example report 1007, in 87.4% of the time (i.e., 497 minutes), the SpO2 values were within 94% and 100%, and in 12% of the time (i.e., 68 minutes), the SpO2 values were between 88% and 93%. A report having different values can be produced in other embodiments.
5. PR Distribution—The report 1008 in FIG. 10G shows an example of PR distribution. The example report 1008 can be created by the application program on the host device. As shown in the example report 1008, in 58.1% of the time (i.e., 331 minutes), the PR values were within 60 bpm and 80 bpm, and in 39.1% of the time (i.e., 222 minutes), the PR values were between 50 bpm and 59 bpm. A report having different values can be produced in other embodiments.

6. PI Distribution—The report 1009 in FIG. 10H shows an example of PI distribution. The example report 1009 can be created by the application program on the host device. As shown in the example chart, in 64.2% of the time (i.e., 365 minutes), the PI values were within 0.1% and 0.5%, and in 35% of the time (i.e., 199 minutes), the PI values were between 0.01% and 0.1%. A report having different values can be produced in other embodiments.
7. SpO2 Desaturations per Hour—The report 1010 in FIG. 101 shows the number of desaturations per hour from baseline. The example report 1010 can be created by the application program on the host device. According to the example chart 1010, the user had 1.9 desaturations per hour with amplitude greater than 4%. A healthy person will typically have less than 5 or so desaturations per hour with amplitude greater than 4%. These values may increase during exercise, or in situations of high stress, or in high altitude places. A report having different values can be produced in other embodiments.
8. Cumulative Time Percentage with SpO2 Less than Threshold—The Report 1011 in FIG. 10J shows the cumulative time percentages with SpO2 less than threshold. The report 1011 can be created by the application program on the host device. According to the example chart 1011, in 1.5% (i.e., 8.5 minutes) of the time, the user's SpO2 values were less than 90%. A healthy person will typically stay for a very small time percentage with SpO2 less than 90%. This time percentage may increase during exercise, or in situations of high stress, or in high altitude places. A report having different values can be produced in other embodiments.
9. PR Volatility Distribution —The PR Volatility expresses the heart rate short-term variability. In one embodiment, the PR volatility is calculated following the steps described in embodiments depicted in FIG. 10P where the measurement data in block 1017 is a series of instantaneous and sequential measurements of PR and the corresponding distribution is one of the statistics computed in block 1020. Typically, for health adults, higher PR Volatility values are better. Increasing PR Volatility trends are positive, and indicative of positive adaptation and/or increase in fitness. The report 1012 in FIG. 10K shows an example of PR Volatility distribution. The report 1012 can be created by the application program on the host device. As shown in the example chart, over 50% of the time (i.e., 286 minutes), the PR Volatility values were between 0.46 bpm and 1 bpm. A report having different values can be produced in other embodiments.
10. PI Log-Volatility Distribution—The PI Log-Volatility expresses the perfusion short-term variability. In one embodiment, the PI Log-Volatility is calculated following the steps described in the embodiment depicted in FIG. 10O where the measurement data in block 1016 is a series of instantaneous and sequential measurements of PI and the corresponding distribution is one of the statistics computed in block 1020. Typically, for health adults, lower PI Log-Volatility values are better. Decreasing PI Log-Volatility trends are positive, and indicative of positive adaptation and/or decrease in overall stress levels. The report 1013 in FIG. 10L shows an example of PI Log-Volatility distribution. The report 1013 can be created by the application program on the host device. As shown in the example chart, over 52% of the time (i.e., 286 minutes), the PI Log-Volatility values were within 9.4% and 15%. A report having different values can be produced in other embodiments.

Additionally or alternatively, a user may share trends in SpO2, PR, PI, and temperature measurements in a file at any time through the application program on the host device (e.g., Trends 1002 in FIG. 10A). One example of a file is a Comma-Separated Values (CSV) file. The file can be opened and displayed by the host device directly or by most presentation or spreadsheet software, such as EXCEL and NUMBERS. In FIG. 10M, the example CSV file 1014 contains 6 columns. Other embodiments can display different values, a different number of rows, and/or a different number of columns. The columns in the example file 1014 are:
1. Date/Time—Stores date and time of each measurement taken. Measurements can be stored once a second (for 12 h trend storage), once every 2 seconds (for 24 h trend storage), once every 3 seconds (for 36 h trend storage), and once every 4 seconds (for 48 h trend storage). Other embodiments can store the measurements at different times.
2. Product Label Barcode. The 8-digit hexadecimal number that identifies the monitoring device that is used at the corresponding time and date.
3.

SpO2 (%)—SpO2 measurements.
4. PR (bpm)—PR measurements.
5. PI (%)—PI measurements.
6. Temperature (° C.)—Core body temperature measurements.

In some instances, the user may share waveforms (e.g., waveforms 1003 in FIG. 10A) through the application program on the host device. In one embodiment, the waveforms are shared in the form of raw data, auxiliary variables, and parameters collected by the monitoring device (up to a certain number of hours) and stored in the host device. Other embodiments can share the waveforms in a different form. The data can be stored into a database file (e.g., WaveformsDB.db) using an encoded data format. The data can be used for in-depth technical analysis of potential problems detected by users, or for off-line computations of other parameters of interest, such as heart rate variability, respiration rate, etc., or to recalculate off-line the values of SpO2, PR, PI, temperature, photoplethysmographs, and ECG waveforms using custom algorithms that may be more suitable for a particular off-line application.

FIG. 10N depicts an example format of a file 1015. The file 1015 includes three columns: Index, Time Stamp, and Data. The Index column indexes the rows of the file 1015. The Time Stamp column has an example format YYYY-MM-DD hh:mm:ss.uuu, where YYYY-MM-DD is the year, month, and day, hh:mm:ss.uuu is the time, in hours, minutes, seconds, and milliseconds, and is the date and time the measurement data was saved to the file 1015. Each time stamp values are an approximation of the time the measurement data was actually collected, since the measurement data is saved by the host device to the file 1015 in real-time. The time stamp values can be used to synchronize in time the measurement data stored in the file 1015 with other measurement systems so as to perform analyses where such a time synchronization is used. The Data column is the actual data saved to the file 1015.

In FIG. 10N, the Data column has three arrays of example data separated by square brackets ([first data array] [second data array] [third data array]. Each of the three arrays has forty samples per row. The samples of each data array are collected synchronously by the monitoring device at every twenty milliseconds (50 Hz sampling frequency), transmitted wirelessly to the host device that stores the data. In one embodiment, the host device stores the data in real-time to temporary buffers (in order to reduce latency requirements) and then saves the data in the buffers to the file 1015 every N seconds, where N is a whole or fractional number greater than zero. For example, the data can be stored to the file 1015 every three-fourths of a second. The data arrays contain the raw waveform data from the one or more light sources in the optical sensor (e.g., red, infrared, and green light sources), and the monitoring device hardware diagnostics variables and parameters, such as, for example, the battery voltage, SoC temperature, current settings for the one or more light sources of the optical sensor, gains for the optical, temperature, and ECG frontends, SoC usage time counters, SoC standby timer counters, ambient light intensity detected by the optical frontend, device identification number, and the like.

In some embodiments, to increase the battery life of the host device (e.g., a smart watch or smart phone), the waveforms (e.g., waveforms 1003 in FIG. 10A) may not be stored on the host device. In this situation, the user may disable the storage. FIG. 12B shows an example embodiment of the setting screen of the application program on the host device, where the user may enable or disable 1206 the storage of the last twelve hours of waveform data.

There are situations where it is desirable to take screenshots of the measurement gauges and trend data for the user's records or for sharing with third parties. Typically, the host device offers this functionality as a standard feature. For instance, in the case of an iOS device from Apple, Inc., the user presses and holds the top (or side) button and one of the volume buttons concurrently to capture a screenshot of the display. The measurement screenshot can be displayed on the display of the iOS device and/or shared by the iOS device.

FIG. 10O illustrates a flowchart of an example method of computing the log-volatility of the measurement data. The process shown in FIG. 10O, for example, can produce the PI Log-Volatility Distribution depicted in FIG. 10L. The method depicted in FIG. 10P, for example, can produce the PR Volatility Distribution depicted in FIG. 10K. In FIG. 10O, the measurement data in a measurement data stream is normalized. In a non-limiting example, the measurement data is normalized by determining the natural logarithm of the measurement data (block 1016). For instance, the measurement data stream can be the PI, PR, or the SpO2 measurement data streams.

At block 1017, the normalized measurement data is filtered to produce a symmetric or asymmetric data stream (around the origin) representing the normalized variability of the original measurement data stream. In one embodiment, a band-pass filter is used to filter the normalized measurement data. At block 1018, the absolute value of the measurement data is determined to turn all of the variability values positive. The measurement data is filtered at block 1019 to obtain the Log-Volatility data stream. In one embodiment, the measurement data is filtered by a low-pass filter for averaging purposes. The resulting measurement data represents the Log-Volatility over time of the original measurement data stream. Statistical analyses (block 1020) can be applied to the resulting measurement data to determine, for example, volatility metrics and/or probability distributions.

FIG. 10P depicts a flowchart of an example method of computing the volatility of the measurement data. Unlike the resulting data produced by the method of FIG. 10O, the volatility measures, produced by the embodiments in FIG. 10P, have the same unit as the original measurement data stream. The method of FIG. 10P is the same process as the method of FIG. 100 except for block 1016, which is omitted in FIG. 10P. Thus, non-normalized measurement data is filtered at block 1021. In some embodiments, blocks 1018 and 1019 can be omitted from the method in FIG. 10P, and statistical analyses (block 1020) is performed after block 1021.

In embodiments where blocks 1018 and 1019 are omitted, the data may have positive and negative values. The resulting statistics are then computed in block 1020 taking into account that the interlining probability distributions are symmetric or asymmetric around the origin. For instance, in some embodiments, the mean value may be zero while the second (e.g., variance, standard deviation), or higher moments may not because block 1018 is not used. Therefore, for embodiments where blocks 1018 and 1019 are omitted, second or higher order moments may be more appropriated to represent the underling volatility or variability of the data. Also, in some instances, The PR Volatility Distribution and the PI Log-Volatility Distribution depicted in FIG. 10K and FIG. 10L respectively, can be replaced with reports depicting the PR Volatility and the PI Log-Volatility over time.

FIG. 10Q illustrates a flowchart of an example method of computing the Ln log-volatility of the measurement data. The illustrated method is a generalization of the methods shown in FIG. 10O. As such, some of the blocks in FIG. 10O are in the process shown in FIG. 10Q and are not described in more detail for brevity.

Initially, as shown in block 1016, the measurement data in a measurement data stream is normalized. In a non-limiting example, the measurement data is normalized by determining the natural logarithm of the measurement data (block 1016). The normalized measurement data is filtered to produce a symmetrical data stream (around the origin) representing the normalized variability of the original measurement data stream (block 1017). At block 1018, the absolute value of the measurement data is determined to change all of the variability values positive.

Next, as shown in block 1030, the measurement data is raised to the n-th power. The number n can be any positive number. For n=1, algorithms described in FIG. 10Q become identical to the algorithms described in FIGS. 100. The measurement data is then filtered at block 1019. Next, the n-th root of the data is determined to provide a higher or a lower weight for each measurement data sample, based on the amplitude of the measurement data (block 1031). Block 1022 converts the measurement data back to the same scale as the original measurement data in the measurement data stream. In one embodiment, blocks 1019 and 1030 are performed concurrently. Statistical analyses (block 1020) can be applied to the resulting measurement data to determine, for example, volatility metrics and/or probability distributions.

FIG. 10R depicts a flowchart of a method of computing the Ln volatility of measurement data. The method of FIG. 10R is the same process as the method of FIG. 10Q except for block 1016, which is omitted in FIG. 10R. Thus, non-normalized measurement data is filtered at block 1032. In some embodiments, the method of FIG. 10R can be used to compute the volatility of data streams when the unit of the computed volatility values is the same as the measurement data (no normalization), and when higher or lower weighting via block 1031 is required for each measurement data sample, based on the amplitude of the measurement.

FIG. 11A illustrates an example screen of identification parameters produced by the application program on the host device. The example screen 1028 can include one or more of the following identification parameters:
1. Serial Num—When the monitoring device is connected to the host device, the host device can display an M-digit hexadecimal number in the screen 1028, where M is a number greater than zero. For example, the M-digit hexadecimal number is a 16-digit hexadecimal number. In one embodiment, the M-digit hexadecimal number is the serial number of the monitoring device.
2. Barcode—When the monitoring device is connected to the host device, the host device may display a P-digit hexadecimal number that identifies the monitoring device in the screen 1028, where P is a number greater than zero. For example, the P-digit hexadecimal number is the barcode from the product label (e.g., product label 906 in FIG. 9B). Additionally, the host device can display the barcode in a start screen of the application program and/or in the trend file that is shared by the host device (e.g., data file 1015 in FIG. 10N). In a non-limiting example, the P-digit hexadecimal number is an 8-digit hexadecimal number.
3. Lot Num—When the monitoring device is connected to the host device, the host device can display the manufacturing lot number of the monitoring device in the screen 1028.
4. Exp Date—When the monitoring device is connected to the host device, the host device may display a date after which the monitoring device should not be sold or used in the screen 1028.
5. Model—When the monitoring device is connected to the host device, the host device can display a Q-digit hexadecimal number of the model number of the monitoring device in the screen 1028. Q is a number greater than zero. For example, the Q-digit hexadecimal number is a 4-digit hexadecimal number.
6. Version—When the monitoring device is connected to the host device, the host device may display an R-digit hexadecimal number of the version number of the monitoring device. For example, the R-digit hexadecimal number is a 4-digit hexadecimal number.
7. App Version—When the monitoring device is connected to the host device, the host device can display an S-digit number of the software version. In a non-limiting example, the S-digit number is a three digit number.

FIG. 11B depicts an example screen of hardware diagnostics parameters produced by the application program. The example screen 1029 can include one or more of the following parameters:
1. LED Power—The LED power parameter in the screen 1029 display the power levels (reported at a Time Stamp) for each light source of the optical sensor in the monitoring device. For example, the power levels of light source one (LED1) and light source two (LED2) are presented. The power level of each light source varies between 0 and 100%.

2. Electronic Gain—The electronic gain parameter in the screen 1029 represents the analog frontend electronic gain (reported at Time Stamp) of the monitoring device. The electronic gain varies between 0 and 40 dB.

3. Ambient Light—The ambient light parameter in the screen 1029 represents the ambient light intensity detected by the one or more photodetectors in the optical sensor in the monitoring device (reported at Time Stamp). It varies between 0 and 100%.

4. SoC Temperature—The SoC parameter in the screen 1029 represents the System on Chip (SoC) temperature in the monitoring device. The SoC temperature can be displayed in Celsius (reported at Time Stamp) or Fahrenheit.

5. Battery Voltage—The battery voltage parameter in the screen 1029 represents the battery voltage in Volts (reported at Time Stamp) of the battery in the monitoring device.

6. Standby Time—The standby time parameter in the screen 1029 represents the amount of time the monitoring device has been activate and disconnected from the host device (reported at Time Stamp).

7. Usage Time—The usage time parameter in the screen 1029 represents the amount of time the device 100 has been activate and connected to the host device 105 (reported at Time Stamp).

8. Time Stamp—The time stamp parameter in the screen 1029 is the date and time when the one or more hardware diagnostic parameters are reported.

FIG. 11C depicts an example embodiment on sharing the measurement data with a technical support team of the monitoring device. When the user wants to share the measurement data, the host device displays the screen 1100. In this example embodiment, the user shares the Identification and Hardware Diagnostics parameters and variables via an electronic mail message, although other embodiments are not limited to electronic mail. The user can also add comments regarding the specific problem or issue found in section 1101. Several other methods can be used to share such information with the manufacturer. In addition, depending on the issue, the user may elect to share the file of the waveform data (e.g., file 1015 in FIG. 10N) with the technical support team for in-depth technical analysis of a potential problem detected by the user.

In some applications, the system that includes the monitoring device, the host device, and the application program, can include a built-in an alarm/warning system to keep the user (e.g., a patient) informed and/or safe. FIG. 12A depicts an example embodiment of an alarm/warning system. FIG. 12A illustrates an example screen 1200, where the gauges 1201 can blink in a color (e.g., red) whenever a measurement value crosses an upper or lower preset limit, or whenever the monitoring device stops providing measurement data. Additionally or alternatively, an audible alarms/warning 1203(e.g., voice, sound) is produced and/or a written message 1202 is displayed whenever the measurement value crosses an upper or lower preset limit, or whenever the monitoring device stops providing measurement data.

Example alarm/warning are now described. The example alarms are listed in descending order of priority, although other embodiments are not limited to the illustrated priority order.
1. Monitoring device is not connected to host device—This alarm is issued whenever the monitoring device is not connected to host device. A visual warning can include causing one or more gauges to blink and/or be displayed in a dashed line. An audible warning may include a beep and/or an audible message such as “Device not connected.” This alarm/warning has the highest priority in one embodiment given that the warning indicates the monitoring device is not connected to the host device, may be out-of-range, defective, or turned off.
2. Battery is empty—The warning is issued whenever the battery in the monitoring device is low or empty. A visual warning can include causing a battery icon (e.g., battery icon 1204) to blink and/or be displayed in a dashed line. An audible warning may include a beep and/or an audible message such as “battery is empty or low.” This alarm/warning has the second highest priority in one embodiment. In some embodiments, the measurement gauges are no longer displayed on the host device when the battery on the monitoring device is low. When the battery on the monitoring device is empty, the measurements are not displayed on the host device.
3. Monitoring device is searching for a valid signal—The warning is issued whenever the monitoring device is connected to the host device but measurements are not yet displayed on the host device because the monitoring device is either not correctly placed on subject, or the data collected is within a transient time where there is not enough collected data to produce a reliable set of measurements. This alarm/warning has the third highest priority in one embodiment. A visual warning can include causing one or more measurement gauges to blink and/or be displayed in a dashed line. An audible warning may include a beep and/or an audible message such as “searching for signal.”
4. Wireless connection with host device is not reliable—This warning is issued whenever the monitoring device has an unreliable wireless connection with the host device. In one embodiment, a visual warning causes all of the measurement gauges blink in a color, such as red. An audible warning may include a beep and/or an audible message such as “poor connection.”
5. SpO2 measurement has crossed either upper or lower limits—This alarm is issued whenever the SpO2 value is outside the preset normal limits. In one embodiment, a visual warning causes the SpO2 measurement gauge to blink in a color, such as red and/or be displayed in a different color. An audible warning may include a beep and/or an audible message such as “saturation warning.” In some instances, the upper and/or lower limits of the SpO2 measurements can be defined by the user in a settings screen, as will be described in more detail in conjunction with FIG. 12B.
6. PR measurement has crossed either upper or lower limits—This alarm is issued whenever the PR value is outside the preset normal limits. In one embodiment, a visual warning causes the PR measurement gauge to blink in a color, such as red and/or be displayed in a different color. An audible warning may include a beep and/or an audible message such as “pulse rate warning.” In some instances, the upper and/or lower limits of the PR measurements can be defined by the user in a settings screen, as will be described in more detail in conjunction with FIG. 12B.
7. SpO2 and PR measurements have crossed either upper or lower limits—This alarm is issued whenever both the SpO2 and the PR values are outside the preset normal limits. In one embodiment, a visual warning causes the SpO2 and PR measurement gauges to blink in a color, such as red and/or be displayed in a different color. An audible warning may include a beep and/or an audible message such as “saturation and pulse rate warning.”
8. Battery in host device is low—This warning is issued whenever the battery in the host device is low or empty. In one embodiment, a visual warning causes a message to be displayed, such as “charge host device.” An audible warning may include a beep and/or an audible message such as “charge host device.” In a non-limiting example, the warning is issued when the charge on the battery in the host device is less than 23%, although other embodiments are not limited to this percentage value.

FIG. 12B depicts an example settings screen of the application program. The screen 1205 enables the user to set the alarm/warning limits and audio settings. The selectable control 1206 (e.g., switch) enables/disables audible warnings. The selectable options 1207 enables the user to set a silence time interval (i.e., 30, 60, 90, or 120 seconds). The silence time interval is used to silence a warning for a given period of time and will be activated when an alarm/warning is active and the user touches one of the measurement gauges (i.e., SpO2, PR, PI, or temperature). The warning/alarm will be paused during the silence time interval and then resume automatically after the silence time interval expires. The selectable elements 1208 and 1209 (e.g., slide control elements) enable the user to set the alarm limits for particular measurement gauges. For example, in the illustrated embodiment, the alarm limits can be set for the SpO2, and PR measurement gauges and the selected values are displayed for each upper and lower limit. In one embodiment, the SpO2 and PR alarm/warning limits are initially set to their default values.

In FIG. 12B, the selectable option 1210 (labeled Voice Gap) enables the user to define the periodicity of voice-based measurements. In the example embodiment depicted in FIG. 12B, the periodicity options are every 30, 60, 120 seconds or “never”. For instance, if the user selects the 30-second option, then every 30 seconds the host device will speak to the user through its speech synthesizer, informing the current measurement values. The host device through its audio system, or through an audio system wirelessly or wired connected to the host device (i.e., headphones, speakers, car sound system, etc.), may speak to the user informing, for example, that “SpO2 is one hundred percent, PR is sixty five beats per minute, PI is one percent, and Temperature is thirty six Celsius”. This functionality enables users to shut-off the screen of the host device, or be driving, exercising, etc. while wearing the monitoring device and not having direct access to the display screen on the host device and still able to hear their current measurement values periodically.

If the user does not want to hear the measurement values, then the option “never” in the selectable option 1205 disables the voice-based measurements. In some embodiments, it is possible to set the host device to provide only voice-based measurements whenever one or some of the measurement values change more than a certain threshold value (absolute or relative). For instance, the host device can audibly output the current measurements whenever the value of SpO2 changes more than +/−2 points, or the value of PR changes more than +/−5 bpm, or the value of PI changes more than +/−10%, or the value of temperature changes more than +/−0.3 Celsius, from their last spoken measurement values. Spoken measurements can also be triggered depending on how the rate of change (first derivative), or the rate of change of the rate of change (second derivative), etc. of a particular measurement variable varies over time.

Additionally or alternatively, the user is informed about the measurement tendency (or rate of change) over time since the last spoken measurement. For example, the host device could speak to the user messages such as “SpO2 is ninety four percent and decreasing”, “PR is one hundred beats per minute and stable”, “PI is zero point two percent and increasing”, “Temperature is thirty six Celsius and stable”. The rate of change can also be specified numerically or qualitatively, such as “increasing slowly”, “increasing fast”, “decreasing slowly”, or “decreasing fast”, etc. The type and content of the measurement message, the triggering rule, and the information contained therein depends on the monitoring application and its particular requirements.

In one aspect, the host device 105 is restricted to the application (a single application) with the hardware buttons and access to the application program menu are disabled and protected by a password (or some other form of authentication) to prevent unauthorized users from changing the settings or disabling the application program. In one example, the application program is compliant with the iOS Guided Access mode when the host device is an iOS device. The Guided Access mode temporarily restricts the iOS device to a single application and lets the user control which features of the application are available. The default behavior of the application program during a Guided Access section can be:
1. The application termination is disabled.
2. The application menu is disabled.
3. Portrait and landscape views are enabled.
4. Hardware buttons are disabled (i.e., volume, Sleep/Wake, etc.).
5. The user can silence audible warnings (if enabled and active) for a period of time by taping on any measurement gauge. However, Audible warnings will resume automatically after the silence duration (i.e., 30, 60, 90, or 120 seconds) expires. If required, audible warnings can be permanently disabled before starting a Guided Access section.

The alarm/warning system described in FIGS. 12A-12B can also relay information about an active alarm/warning to a third party via the host device. The host device can send notifications (wirelessly or wired) directly to third parties. Example third parties include, but are not limited to nurses, medical doctors, and caretakers. Additionally or alternatively, the notifications are sent to a central alarm/warning system that in turn, relays the notifications to the appropriate receiver.

The battery is non-rechargeable in some embodiments. Information about the charge on the battery is provided to the user at select times or continuously and/or the device usage to enable the user to predict when monitoring device is to be replaced. The battery icon 1204 shown in FIG. 12A is shown in detail in FIG. 13A. FIG. 13A depicts a battery icon and example battery states (e.g., battery full, not full, low, and empty).

FIGS. 13B-13C illustrate a method to compute the life of the battery in the monitoring device. The method does not use specialized circuitry that can increase the manufacturing costs of the monitoring device. The challenge to accurately estimate the battery life directly from the battery voltage is that the battery voltage changes very little throughout most of its discharge curve, making unreliable the mapping from battery voltage to battery life (or time left).

FIG. 13B illustrates an example battery discharge curve. The battery voltage (x axis) starts from Va (fully charged) and ends in Vc (fully discharged). From Va to Vb, the battery discharge curve 1300 is too vertical to produce an accurate estimate for battery life(y axis) from the battery voltage. The y-axis can represent time, percentage, joules, or another suitable value. The challenge of estimating the battery life is addressed by a hybrid method of estimating the battery life. The hybrid method uses a smart counter in combination with battery voltage measurements to more accurately estimate the battery life.

An example method of estimating the battery life of the battery in the monitoring device is shown in FIG. 13C. The monitoring device is activated and the processing device in the monitoring device (e.g., 102 in FIG. 1) starts a usage counter T (blocks 1310 and 1311). Ta represents the time it takes to have the battery completely discharged when the monitoring device is under operation. For instance, if the battery life has typical value, minimum value, maximum value or average value of 24 hours, then Ta is set to a value that corresponds to 24 hours of battery life.

In one embodiment, the usage counter is implemented in non-volatile memory (e.g., a flash memory) so as not to lose the counter value in the event of a power transient. The usage counter is decremented periodically by the processing device at block 1312 based on the electrical loads in the circuitry of the monitoring device. The higher the power consumption of a particular load, the larger the corresponding decrement (ATi). Some loads are constant over time, and some tend to vary depending on known factors. For instance, the required current of a light source (LED) may vary depending on the measurement site's optical opaqueness. As a result, the corresponding ΔTi of a particular LED should be adjusted by its set current. The higher the current, the higher the ΔTi value.

When the battery voltage drops below Vb (FIG. 13A), the amount of battery life left (e.g., the amount of charge on the battery) is estimated through the function ƒ(Vbat, Tb) at block 1313. The function ƒ(Vbat, Tb) can be implemented in several ways. One implementation for ƒ(Vbat, Tb) is to be the minimum value between the battery life estimate from the battery voltage curve (as depicted in FIG. 13B) and the last (Tb) battery life estimate from the usage counter, with ƒ(Vbat,Tb)=Tb for Vbat=Vb. This ensures functional continuity for the measurements of battery life, even if the battery voltage increases momentarily because of changes in ambient temperature, circuitry loads, etc. The battery is considered fully discharged (empty) when the battery voltage drops below Vc (FIG. 13A) at block 1314. The different stages of the battery life estimate shown in FIG. 13A can be used to inform the user via the battery icon 1204. The stages may also be used to trigger a battery empty alarm/warning when the battery is fully discharged.

The usage counter implemented in block 1311 of FIG. 13C can also be used to monitor the usage time of the monitoring device. In some embodiments, an additional counter (a standby counter) can be used to account for the amount of time the monitoring device is in a standby mode (i.e., activated but not connected to the host device). The standby counter can be implemented in the same non-volatile memory (e.g., flash memory) with the usage counter. FIG. 11B depicts the Usage Time and Standby Time in the screen 1029. The usage and standby times are calculated based on the aforementioned battery and standby counters, which are implemented in the non-volatile memory, and the values of the usage and standby counters are sent wirelessly and periodically to the host device. In one embodiment, because the usage and the standby counters cannot be deleted from the outside, or by an unauthorized user without deleting the whole firmware, thereby rending the monitoring device nonoperational, the usage and the standby counters can be used to disable the monitoring device once the usage and/or the standby counter reach a certain value. This prevents unauthorized users from replacing the non-rechargeable battery (tampering) to enable monitoring device to be used for an extended period of time. The usage and the standby counters can increase the safety of monitoring device because the monitoring device cannot be repaired or disassembled without compromising the circuitry in the monitoring device.

Another safety feature can be implemented by starting a voltage timer or counter (“voltage timer”) whenever the voltage of the battery drops below a certain threshold. If the battery voltage stays below the threshold until the voltage timer reaches a pre-defined value, the battery voltage cannot increase past the threshold given that the battery is non-rechargeable, and a flag is set in the non-volatile memory indicating the battery is fully discharged. Later, in the event the battery voltage becomes greater than the threshold, with the counters (e.g., usage and/or standby) expired, an inference can be made that an unauthorized user (or third-party) has tempered with monitoring device. In this case, the firmware of the monitoring device may reset the processing device (e.g., processing device 102 in FIG. 1) and go to an idle state in order to prevent unauthorized use or repurposing of the monitoring device.

FIG. 13D depicts a flowchart of an anti-tampering method for the monitoring device. Initially, as shown in block 1320, a determination is made as to whether the monitoring device is active and in use. If the monitoring device is in use, the process passes to block 1321 where the usage counter is incremented. A determination is made at block 1322 as to whether the usage counter has reached its maximum value. If so, the method continues at block 1323 where the monitoring device stops operating and cannot be used. In an alternate embodiment, if the usage counter reaches its maximum value, then block 1323 can be executed only after the connection (e.g., wireless connection) between the monitoring device and the host device is lost to stop operation. This ensures that the patient or user is not put at risk due to the lack of operation of the monitoring device because the usage counter has reached its limit (maximum value).

Returning to block 1320, if the monitoring device is not in use, it is assumed the monitoring device is in standby mode and the standby counter is incremented at block 1324. A determination is made at block 1325 as to whether the standby counter has reached its maximum value. If so, the process passes to block 1323 where the monitoring device stops operating and cannot be used. Again, in an alternate embodiment, if the standby counter reaches its maximum value, then block 1323 can be executed only after the connection (e.g., wireless connection) between the monitoring device and the host device is lost to stop operation. This ensures that the patient or user is not put at risk due to the lack of operation of the monitoring device because the standby counter has reached its limit (maximum value).

When the standby counter and the usage counter have not reached the maximum values, the method continues at block 1326 where a determination is made as to whether the battery voltage is below a pre-defined voltage threshold. If so, a threshold counter is incremented at block 1327. Otherwise, the threshold counter is reset at block 1328.

A determination is made at block 1329 as to whether the threshold counter has reached its maximum value. If so, the method passes to block 1330 where the monitoring device either stops operation (e.g., immediately), continues normal operation until the standby or usage counters expire, or stops operation after the battery voltage increases higher than the pre-defined voltage threshold. The method returns to block 1320 when the threshold counter has not reached its maximum value.

In some embodiments, the standby, usage and threshold counters are implemented in the non-volatile memory in the monitoring device. Alternatively, the threshold counter can be implemented in the volatile memory in the monitoring device, or in the volatile memory of the host device, given that the threshold counter is typically used to measure time intervals much smaller than typical usage or standby time intervals. Implementing the counters in the non-volatile memory decreases the chances of an occurrence of power outage, failure, or reset that may cause the counters to lose their current values.

FIG. 14A depicts a first example method of determining an active noise floor of a monitoring device. Initially, the monitoring device is turned on at block 1400. For example, in one embodiment the monitoring device is turned on by removing the first tab and pressing the indicator (e.g., tab 909 and indicator 910 in FIG. 9D). In block 1402, the monitoring device is connected (e.g., wirelessly connected) to the host device. In one embodiment, the monitoring device is paired with the host device using BLUETOOTH.

Once the connection between the monitoring device and the host device is established, an active noise floor measurement is performed on the host device (block 1404). In a non-limiting example, the monitoring device is placed in a dark environment (e.g., limited or no ambient light) for the active noise floor measurement. In another non-limiting example, the monitoring device modulates the light source signals and/or demodulates the received optical signals in order to enable active noise floor measurements under ambient light and/or electromagnetic interferences. The modulation and demodulation algorithms prevent ambient light signals and/or electromagnetic interferences from interfering with the active noise floor measurement. The noise floor measurement is used to quantify the signal-to-noise ratios and metrics that quantify the performance of the monitoring device prior to placing the monitoring device on a measurement site. The noise floor measurement is useful in applications that the manufacturing, storage, shipping and/or handling processes may cause the monitoring device to become out of specifications due to unforeseeable events. In an example embodiment, the noise floor measurement is generated by activating the one or more light sources (e.g., 113 in FIG. 1) in the monitoring device as if the monitoring device is in normal operation. The light will diffuse through the material in the second tab (tab 911) and reach the one or more photodetectors via diffusive and/or specular reflectance. The signals produced by the photodetector(s) (e.g., 114 in FIG. 1) are collected by the monitoring device and are demodulated, filtered, processed and sent to the host device for computation of the signal-to-noise ratios and metrics.

In block 1406, a determination is made as to whether the computed signal-to-noise ratios and/or metrics are acceptable (e.g., greater than a predefined threshold). If not, the host device indicates to the user that the monitoring is non-operational or defective and operations stop (block 1408). In an alternate embodiment, multiple noise floor runs can be acquired and processed before block 1406 is performed.

When the computed signal-to-noise ratios and/or the metrics are acceptable, the process passes to block 1410 where the monitoring device is attached to a measurement site. For example, the second tab (e.g., tab 911) is removed to expose the adhesive material and attach the monitoring device to a measurement site. The monitoring device and the host device then enter normal monitoring operations (block 1412).

FIG. 14B illustrates a second example method of determining an active noise floor of a monitoring device. In the illustrated embodiment, the noise floor calculations are performed by the monitoring device prior to connecting with host device. Initially, the monitoring device is turned on at block 1400. For example, in one embodiment the monitoring device is turned on by removing the first tab and pressing the indicator (e.g., tab 909 and indicator 910 in FIG. 9D).

An active noise floor measurement is obtained by the monitoring device at block 1414. The noise floor measurement is determined by activating the one or more light sources in the monitoring device as if the monitoring device is in normal operations (e.g., light source(s) 113 in FIG. 1). The light will diffuse through the material in the second tab (e.g., tab 911 in FIG. 9D) and be detected by the one or more photodetectors via diffusive and/or specular reflectance (e.g., photodetector(s) 114 in FIG. 1). The signals produced by the photodetector(s) are demodulated, filtered, and processed by the monitoring device to compute the signal-to-noise ratios and metrics.

A determination is then made at block 1416 as to whether the computed signal-to-noise ratios and/or metrics are acceptable (e.g., greater than predefined thresholds). If the computed signal-to-noise ratios and/or metrics are not acceptable, the operations end at block 1408. In one embodiment, the monitoring device indicates to the user that the monitoring device is non-operational or defective (e.g., by producing a flashing light(s) in a particular pattern or mode using at least one light source in the monitoring device). In an alternate embodiment, multiple noise floor runs can be acquired and processed before block 1416 is performed.

When the computed signal-to-noise ratios and/or metrics are acceptable, the process passes to block 1410, where the monitoring device is attached to a measurement site. In one embodiment, the second tab (e.g., tab 911 in FIG. 9D) is removed to expose the adhesive and the monitoring device is attached to a measurement site. The monitoring device is then connected (e.g., wirelessly connected) to the host device at block 1402. The monitoring device and the host device enter normal monitoring operations at block 1412.

FIG. 15A depicts an example battery discharge curve. The x-axis represents the battery voltage and the y-axis can represent time, percentage, joules, voltage or other values. The example battery discharge curve is divided into three charge-voltage regions 1502, 1504, 1506. In the first charge-voltage region 1502, the battery is fully charged or almost fully charged. In region 1502, small changes (drops) in the battery charge create relatively large changes (drops) in the battery voltage, which are observable. As a result, it is possible to determine the battery charge (e.g., battery life) from the battery voltage and/or other available parameters of interest (e.g., ambient temperature, circuitry loads, etc.).

In the second charge-voltage region 1504, changes in battery charge do not affect (or substantially affect) the battery voltage. As a result, in the second charge-voltage region 1504, the battery charge is accounted for via one or more counters (or timers). In other words, region 1504 does not include observable or measurable parameters that can be used to estimate the battery charge directly. Thus, the one or more counters and/or information about other available parameters of interest (e.g., ambient temperature, circuitry loads, etc.) is used to estimate the battery charge.

As the battery discharges, the battery enters the third charge-voltage region 1506 where the battery charge can again be estimated from the observable battery voltage and/or other available parameters of interest (e.g., ambient temperature, circuitry loads, etc.). In an alternate embodiment, first and the second regions 1502, 1504 can be combined into a single first/second non-observable charge-voltage region 1508 and the calculations are performed as described for region 1504.

FIGS. 15B-15C illustrate example methods of estimating the battery life of the battery in the monitoring device. The computations in each method are performed in both a closed loop and an open loop via different functions. In the closed loop, the battery voltage and/or other available parameters of interest (e.g., ambient temperature, circuitry loads, etc.) is used to estimate the battery charge directly. In the open loop, the battery charge is estimated indirectly using one or more counters and/or other available parameters of interest (e.g., ambient temperature, circuitry loads, etc.).

In FIG. 15B, the battery in in the monitoring device is operating in the first charge-voltage region at block 1510 (e.g., 1502 in FIG. 15A). In an example embodiment, the battery charge is calculated by a function that maps the battery voltage and other parameters of interest (i.e., voltage, temperature, circuitry loads, etc.) into normalized battery charge values. In one embodiment, the monitoring device measures the battery voltage and the other parameters of interest and transmits the measurements to the host device. The host device determines the normalized battery charge values.

In block 1512, the battery charge reaches the second charge-voltage region (e.g., 1504 in FIG. 15A) and the battery charge is estimated using one or more counters that are implemented in the monitoring device (e.g., in non-volatile memory). The counter value(s) are transmitted to the host device for processing and the host device decreases the battery charge over time as a function of the counter value(s). In one embodiment, the one or more counters represent different operating modes in the monitoring device. In an example embodiment, a first counter and a second counter can be implemented in the monitoring device. The first counter (“standby counter”) is incremented whenever the monitoring device is in standby mode (e.g., not connected to the host device). The second counter (“usage counter”) is incremented whenever the monitoring device is connected to the host device and in operation. The standby and usage modes can each have a different power consumption level, and as a result, the different standby and usage counters are used to record values that are proportional to the energy consumption of the monitoring device. The standby and usage counters can be incremented (or decremented) based on elapsed time and/or other available parameters of interest (e.g., ambient temperature, circuitry loads, etc.) to account for the battery discharging over time. The values of the standby and usage counters are mapped by the host device into normalized battery energy values whenever the battery is operating in the second charge-voltage region 1504.

Next, as shown in block 1514, the battery is operating in the third charge-voltage region (e.g., region 1506 in FIG. 15A), and similar to the first charge-voltage region, the battery charge is calculated by a function that maps the battery voltage and other parameters of interest (i.e., voltage, temperature, circuitry loads, etc.). The battery remains in the third charge-voltage region until the battery discharges completely. When the battery is discharged fully, the host device can produce warnings and notification that the charge on the battery in the monitoring device is low or empty.

FIG. 15C depicts a flowchart of an alternate method of estimating the battery life of the battery in the monitoring device. In FIG. 15C, the first and the second charge-voltage regions (e.g., regions 1502 and 1504 in FIG. 15A) are combine into a combined charge-voltage region (1508 in FIG. 15A). The combined region reduces the number of charge-voltage regions and simplifies the operations of estimating the battery life. Typically, the battery is in the first charge-voltage region for a shorter period of time than in region 1504. Thus, combining the first and the second charge-voltage regions into one combined region does not affect significantly the accuracy of the estimations.

Initially, as shown in block 1516, the battery in the monitoring device is in the first/second charge-voltage region and the battery charge is estimated using the one or more counters that are implemented in the monitoring device. The counter value(s) are transmitted to the host device for processing. The host device decreases the battery charge over time as a function of the counter value(s). In a non-limiting embodiment, the counter(s) are implemented as the usage and the standby counters. The values of the standby and usage counters are mapped by the host device into normalized battery energy values whenever the battery is operating in the combined region.

Next, as shown in block 1514, the battery is operating in the third charge-voltage region and the battery charge is calculated by a function that maps the battery voltage and other parameters of interest (i.e., voltage, temperature, circuitry loads, etc.) into normalized battery energy values. The battery remains in the third charge-voltage region until the battery discharges completely. When the battery is discharged fully, the host device can produce warnings and notification that the charge on the battery in the monitoring device is low or empty.

The system of the monitoring device and the host device is a hybrid or distributed system. Some of the functionalities are implemented in the monitoring device and some of the functionalities are implemented in the host device. For example, in one embodiment, the monitoring device implements the counter(s) and measures/calculates one or more parameters of interest (i.e., voltage, temperature, circuitry loads, etc.). The monitoring device transmits the values to the host device, where the host device performs the open loop and closed loop fuel gauge calculations, displays battery fuel gauge icon (e.g., see FIG. 13A) and issues warnings and/or notifications whenever the charge on the battery is low or empty.

FIG. 16 illustrates a flowchart of a method of operating a monitoring device. Initially, as shown in block 1600, the monitoring device is turned on and an indicator light indicates the monitoring device is in standby mode. The standby mode represents a mode in which the monitoring device is waiting to be operably connected to a host device (e.g., wirelessly connected to the host device). In one embodiment, a connection timer begins, where the connection timer monitors (e.g., counts down) a given time period in which the monitoring device attempts to connect to the host device.

The monitoring device then attempts to connect to the host device at block 1602. In one embodiment, the monitoring device repeatedly attempts to connect to the host device until the monitoring device successfully connects to the host device or until the connection timer times out (e.g., expires). When the monitoring device successfully connects to the host device, process passes to block 1606 where the monitoring device begins operations. In some embodiments, the connection timer is a watchdog timer that is reinitialized at select times or periodically by the monitoring device firmware whenever the monitoring device is in normal operation. This ensures that the monitoring device processor and circuitry are only reset if a software and/or hardware fault occurs and the connection timer (i.e., watchdog timer) times out. If the monitoring device is in standby mode, then the connection timer (i.e., watchdog timer) will time out at select times or periodically, forcing the monitoring device to reset itself. This ensures that the monitoring device will be always operational (i.e., in standby or connected) and eliminates completely the need for reset and/or power switches in the monitoring device circuitry in order to make unnecessary manual resets or power up sequences by the user in the event of a software or hardware fault. When the monitoring device is not able to connect to the host device in block 1602 within the given time period or the monitoring device loses connection with the host device in block 1606 due to hardware or software fault, the connection timer (i.e., watchdog timer) times out, and the method continues at block 1604 where a reset operation on the monitoring device is performed. The monitoring device processor and circuitry are reset, and indicator light turned off After short period of time, the monitoring device processor and circuitry are reinitialized, the indicator light turned on in block 1600, and the process in FIG. 16 repeats itself.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternative aspects falling within the spirit of the broader aspects of the general inventive concept embodied in this application that do not depart from the broader scope of the claimed disclosure.

What is claimed is:

1. A monitoring device, comprising:
an optical sensor within an enclosure of the monitoring device and comprising a light source and a photodetector positioned adjacent a first surface of the enclosure, wherein the light source is operable to emit light towards the measurement site and the photodetector is operable to receive light reflected from the measurement site when the first surface is in contact with a measurement site of a first body part of a user;
a temperature sensor within the enclosure of the monitoring device and positioned adjacent the first surface of the enclosure and operable to measure a temperature at the measurement site when the first surface is in contact with the measurement site;
a first electrical contact sensor within the enclosure of the monitoring device and positioned adjacent the first surface of the enclosure to be in contact with the measurement site when the first surface is in contact with the measurement site;
a second electrical contact sensor within the enclosure of the monitoring device and positioned adjacent a second surface of the enclosure, wherein the first and the second electrical contact sensors detect heart signals when a different second body part of the user contacts the second electrical contact sensor; and
a wireless communication device operable to transmit signals received from the photodetector, temperature measurements, and the heart signals to an application program on a host device.

2. The monitoring device of claim 1, further comprising a processing device operably connected to the optical sensor, the temperature sensor, and the first and the second electrical contact sensors and operable to process higher frequency, lower latency or lower complexity computations using the signals received from the photodetector, temperature measurements, or the heart signals and the application program on the host device is operable to process lower frequency, higher latency, or higher complexity computations.

3. The monitoring device of claim 1, wherein the measurement site is an ear of the user.

4. The monitoring device of claim 1, wherein the measurement site is a finger of the user.

5. The monitoring device of claim 1, wherein the measurement site is a forehead of the user.

6. The monitoring device of claim 1, wherein the monitoring device is incorporated into a hat worn by the user.

7. The monitoring device of claim 6, wherein:
the hat further comprises a plurality of electroencephalogram (EEG) electrodes; and
the wireless communication device is operable to transmit signals from the plurality of EEG electrodes to the application program on the host device.

8. The monitoring device of claim 1, wherein the monitoring device is incorporated into a patch attached to the measurement site.

9. A system, comprising:
a monitoring device, comprising:
an optical sensor within an enclosure of the monitoring device and comprising a light source and a photodetector positioned adjacent a first surface of the enclosure, wherein the light source is configured to emit light towards the measurement site and the photodetector is configured to receive light reflected from the measurement site when the first surface is in contact with a measurement site of a first body part of a user;
a temperature sensor within the enclosure of the monitoring device and positioned adjacent the first surface of the enclosure and configured to measure a temperature at the measurement site when the first surface is in contact with the measurement site;
a first electrical contact sensor within the enclosure of the monitoring device and positioned adjacent the first surface of the enclosure to be in contact with the measurement site when the first surface is in contact with the measurement site;
a second electrical contact sensor within the enclosure of the monitoring device and positioned adjacent a second surface of the enclosure, wherein the first and the second electrical contact sensors detect heart signals when a different second body part of the user contacts the second electrical contact sensor; and
a wireless communication device operable to transmit signals received from the photodetector, temperature measurements, and the heart signals; and
an application program on a host device operable to:
process the signals transmitted by the monitoring device to compute a physiological parameter, waveform data associated with the physiological parameter, and trend data associated with the physiological parameter; and
cause the physiological parameter and at least one of a waveform associated with the physiological parameter or a trend associated with the physiological parameter to be displayed.

9. The system of claim 9, wherein:
the application program generates a first alarm when the physiological parameter exceeds an upper limit; and
the application program generates a second alarm when the physiological parameter is less than a lower limit.

11. The system of claim 10, wherein the upper limit and the lower limit are set in a settings user interface of the application program.

12. The system of claim 10, wherein the physiological parameter is one of a pulse rate, a perfusion index, or blood oxygen saturation.

13. The system of claim 10, wherein the first processing device is operable to process higher frequency, lower latency or lower complexity computations using the signals received from the photodetector, temperature measurements, or the heart signals and the application program on the host device is operable to process lower frequency, higher latency, or higher complexity computations.

14. The system of claim 10, wherein the measurement site is an ear of the user.

15. The system of claim 10, wherein the measurement site is a finger of the user.

16. The system of claim 10, wherein the measurement site is a forehead of the user.

17. The system of claim 10, wherein the application program causes a screen to be displayed that enables the user to share the physiological parameter, the physiological parameter or trend data with another computing device.

18. The system of claim 10, wherein the application program:
causes a screen to be displayed that enables the user to select a report to be shared with another computing device, the report comprising analysis data of the waveform data or the trend data associated with the physiological parameter; and
based on a selected report, generates the report and causes the report to be transmitted to the other computing device.

19. The system of claim 18, wherein the report comprises one of:
blood oxygen saturation measurements over time;
blood oxygen saturation distribution;
pulse rate measurements over time;
pulse rate distribution;
pulse rate volatility distribution;
perfusion index measurements over time;
perfusion index distribution; or perfusion index log-volatility distribution.

20. The system of claim 10, wherein:
the monitoring device is incorporated into a hat worn by the user; or the monitoring device is incorporated into a patch that attaches to the measurement site.
